(19) United States
(12) Patent Application Publication
(10) Pub. No.: US 2023/0093471 A1
(43) Pub. Date: Mar. 23, 2023

(54) METHODS AND SYSTEMS FOR PREDICTING RATES OF PROGRESSION OF AGE-RELATED MACULAR DEGENERATION

(71) Applicant: The United States of America, as represented by the Secretary, Dept. of Health and Human Services, Bethesda, MD (US)

(72) Inventors: Emily Ying Chew, Bethesda, MD (US); Zhiyong Lu, Bethesda, MD (US); Tiarnan Daniel Lenaghan Keenan, Bethesda, MD (US); Wai T. Wong, Bethesda, MD (US); Yifan Peng, Bethesda, MD (US); Qingyu Chen, Bethesda, MD (US); Elvira Agrón, Bethesda, MD (US)

(21) Appl. No.: 17/904,573

(22) PCT Filed: Feb. 18, 2021
(86) PCT No.: PCT/US2021/018589

§ 371 Date: Aug. 18, 2022

Related U.S. Application Data
(60) Provisional application No. 62/978,070, filed on Feb. 18, 2020.

(51) Int. Cl. G16H 50/30; C12Q 1/6883; G06T 7/00

(52) U.S. Cl. CPC G16H 50/30; C12Q 1/6883; G06T 7/0012; G06T 2207/30041

(57) ABSTRACT

Disclosed herein are systems and methods for predicting risk of late age-related macular degeneration (AMD). The method may include receiving one or more color fundus photograph (CFP) images from both eyes of a patient, classifying each CFP image, and predicting the risk of late AMD by estimating a time to late AMD. Classifying each CFP image may include extract one or more deep features for macular drusen and pigmentary abnormalities in each CFP image, grading the drusen and pigmentary abnormalities and/or detecting the presence of RPD in each CFP image. Predicting the risk of late AMD may include estimating a time to late AMD using a Cox proportional hazard model using the presence of RPD, the one or more deep features, and/or the graded drusen and pigmentary abnormalities.

### GOVERNMENT INTEREST STATEMENT

The present subject matter was made with U.S. government support. The U.S. government has certain rights in this subject matter.

### FIELD

The present disclosure relates to systems and methods for predicting rates of progression of age-related macular degeneration (AMD). More specifically, the disclosure relates to predicting rates of progression of AMD and determining the presence of reticular pseudodrusen (RPD) using color fundus photography.

### BACKGROUND

By 2040, age-related macular degeneration (AMD) will affect approximately 288 million people worldwide. Identifying individuals at high risk of progression to late AMD, the sight-threatening stage, is critical for clinical actions, including medical interventions and timely monitoring. Although deep learning has shown promise in diagnosing/screening AMD using color fundus photographs (CFP), it remains difficult to predict individuals' risks of late AMD and presence of RPD accurately. For both tasks, these initial deep learning attempts have remained not fully validated in independent cohorts.

The ability to detect RPD presence accurately but accessibly is clinically important for multiple reasons. RPD are now recognized as an important AMD lesion. Their presence is strongly associated with increased risk of progression to late AMD. Identifying these eyes with high likelihood of progression is essential so that clinicians can intervene in a timely way to decrease risk of visual loss. These clinical interventions include prescribing medications (e.g. AREDS2 oral supplements), smoking cessation, dietary interventions, tailored home monitoring, and tailored reimaging regimens. Importantly, RPD presence is suggested as the critical determinant of the ability of subthreshold nanosecond laser that may decrease progression from intermediate to late AMD. However, current attempts to incorporate this key lesion into AMD classification and risk prediction algorithms are hampered. Since RPD grading requires access to both multi-modal imaging and expert graders, ascertainment is limited to the research setting in specialist centers only.

Therefore, there is a need for a method of predicting rates of progression of AMD and also for determining the presence of RPD. Since multi-modal imaging is not typically performed in routine clinical practice, the ability to detect AMD and predict progression is important. RPD presence is a predictor of progression thus detection of RPD from CFP alone would represent a valuable step forward in accessibility.

### SUMMARY

This disclosure provides a method of predicting risk of late age-related macular degeneration (AMD). In some aspects, the method may include receiving one or more color fundus photograph (CFP) images from both eyes of a patient and classifying each CFP image. Classifying each CFP image may include extracting one or more deep features in each CFP image, or grading drusen and pigmentary abnormalities. The method may further include predicting the risk of late AMD by estimating a time to late AMD using a Cox proportional hazard model using the one or more deep features or the graded drusen and pigmentary abnormalities.

The disclosure further provides a method of predicting risk of late AMD. The method may include receiving one or more images from both eyes of a patient and classifying each image. Classifying each image may include detecting the presence of RPD in each image. The method may further include predicting the risk of late AMD by estimating a time to late AMD using the presence of RPD in each image.

Also provided herein is a device having at least one non-transitory computer readable medium storing instructions which when executed by at least one processor, cause the at least one processor to: receive one or more color fundus photograph (CFP) images from both eyes of a patient, classify each CFP image, and predict the risk of late AMD. In some aspects, classifying each CFP image may include extracting one or more deep features in each CFP image; grade the drusen and pigmentary abnormalities; and/or detecting the presence of RPD in each CFP image. Predicting the risk of late AMD may include estimating a time to late AMD using a Cox proportional hazard model using the presence of RPD, the one or more deep features, and/or the graded drusen and pigmentary abnormalities.

### BRIEF DESCRIPTION OF THE DRAWINGS

The description will be more fully understood with reference to the following figures and data graphs, which are presented as various embodiments of the disclosure and should not be construed as a complete recitation of the scope of the disclosure. It is noted that, for purposes of illustrative clarity, certain elements in various drawings may not be drawn to scale. Understanding that these drawings depict only exemplary embodiments of the disclosure and are not therefore to be considered to be limiting of its scope, the principles herein are described and explained with additional specificity and detail through the use of the accompanying drawings in which:

FIG. 1 is a flowchart of the two-step architecture of the method of predicting risk of late AMD.

FIG. 2 illustrates the creation of the study data sets. To avoid ‘cross-contamination’ between the training and test datasets, no participant was in more than one group.

FIG. 3 shows prediction error curves of the survival models in predicting risk of progression to late age-related macular degeneration on the combined AREDS/AREDS2 test sets (601 participants), using the Brier score (95% confidence interval).

FIG. 4 illustrates example system embodiments.

FIG. 5 illustrates an example machine learning environment.

FIG. 6A shows a screenshot of an example software for implementing the late AMD risk prediction method.

FIG. 6B shows four selected color fundus photographs with highlighted areas used by the deep learning classification network (DeepSeeNet). Saliency maps were used to represent the visually dominant location (drusen or pigmentary changes) in the image by back-projecting the last layer of neural network.

FIG. 7 is an overview of training a deep learning method for detecting RPD.

FIG. 8A shows receiver operating characteristic curves of five different deep learning convolutional neural networks for the detection of reticular pseudodrusen from fundus autofluorescence images, using the full test set.

FIG. 8B shows receiver operating characteristic curves of five different deep learning convolutional neural networks for the detection of reticular pseudodrusen from the corresponding color fundus photographs, using the full test set.

FIG. 9A shows receiver operating characteristic curves for the detection of reticular pseudodrusen by the convolutional neural network DenseNet from fundus autofluorescence images. The performance of the four ophthalmologists on the same test sets is shown by four single points. In all cases, the ground truth is the reading center grading of the fundus autofluorescence images.

FIG. 9B shows receiver operating characteristic curves for the detection of reticular pseudodrusen by the convolutional neural network DenseNet from the corresponding color fundus photographs. The performance of the four ophthalmologists on the same test sets is shown by four single points and the performance of the reading center grading of the color fundus photographs is also shown as a single point. In all cases, the ground truth is the reading center grading of the fundus autofluorescence images.

FIG. 10A, FIG. 10B, and FIG. 10C show deep learning attention maps overlaid on fundus autofluorescence (FAF) images and color fundus photographs (CFP). For each of the three representative eyes, the FAF image (left column) and CFP (right column) are shown in the top row, with the corresponding attention maps overlaid in the bottom row. The heatmap scale for the attention maps is also shown: signal range from −1.00 (purple) to +1.00 (brown).

FIG. 11 shows an example framework for multi-modal, multi-task, multi-attention (M3) deep learning convolutional neural network (CNN) for the detection of reticular pseudodrusen from color fundus photographs (CFP) alone, their corresponding fundus autofluorescence (FAF) images alone, or the CFP-FAF image pairs.

FIG. 12 shows box plots showing the F1 score results of the multi-modal, multi-task, multi-attention (M3) and standard (non-M3) deep learning convolutional neural networks for the detection of reticular pseudodrusen from color fundus photographs (CFP) alone, their corresponding fundus autofluorescence (FAF) images alone, or the CFP-FAF image pairs, using the full test set. The horizontal line represents the median F1 score and the boxes represent the first and third quartiles. The whiskers represent quartile 1−(1.5×interquartile range) and quartile 3+(1.5×interquartile range). The dots represent the individual F1 scores for each model. ****: P≤0.0001; ***: P≤0.001 (Wilcoxon rank-sum test). Note that the Y-axis of the CFP scenario is different.

FIG. 13 is a differential performance analysis: distribution of test set images correctly classified by both models, neither model, the multi-modal, multi-task, multi-attention (M3) model only, or the non-M3 model only, for the detection of reticular pseudodrusen from color fundus photographs (CFP) alone, their corresponding fundus autofluorescence (FAF) images alone, or the CFP-FAF image pairs, using the full test set.

FIG. 14A shows deep learning attention maps overlaid on representative image examples for color fundus photographs (CFP) alone; FIG. 14B shows deep learning attention maps overlaid on representative image examples for fundus autofluorescence (FAF) images alone; and FIG. 14C shows deep learning attention maps overlaid on representative image examples for the CFP-FAF image pairs for the detection of reticular pseudodrusen (RPD) by the multi-modal, multi-task, multi-attention (M3) model or the non-M3 model: representative examples where the non-M3 model missed RPD presence but the M3 model correctly detected it. For each image, the attention maps demonstrate quantitatively the relative contributions made by each pixel to the detection decision. The heatmap scale for the attention maps is also shown: signal range from −1.00 (purple) to +1.00 (brown). RPD are observed on the FAF images as ribbon-like patterns of round and oval hypoautofluorescent lesions with intervening areas of normal and increased autofluorescence. Areas of RPD clearly apparent to human experts are shown (black arrows), as well as areas of RPD possibly apparent to human experts (dotted black arrows).

FIG. 15A and FIG. 15B show receiver operating characteristic (ROC) curves of the multi-modal, multi-task, multi-attention (M3) and standard (non-M3) deep learning convolutional neural networks for the detection of reticular pseudodrusen from color fundus photographs (CFP) alone (FIG. 15A) or their corresponding fundus autofluorescence (FAF) images alone (FIG. 15B), using a random subset of the test set. The mean ROC curve is shown (dotted line), together with its standard deviation (shaded area). The performance of the 13 ophthalmologists on the same test sets is shown by 13 single points.

FIG. 16 shows an example adversarial attack, consisting of an original CFP image and adversarial perturbations generated by projected gradient descent (PGD) 16 with 20 steps, the maximal pixel perturbation=1 and the attack step size=4. ϵ denotes the maximum number of pixels modified.

FIG. 17 shows the framework's flowchart for adversarial training and testing adversarial attack examples. Inter-denoising and intra-denoising layers follow the ReLU layer.

Reference characters indicate corresponding elements among the views of the drawings. The headings used in the figures do not limit the scope of the claims.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and, such references mean at least one of the embodiments.

Reference to “one embodiment”, “an embodiment”, or “an aspect” means that a particular feature, structure, or characteristic described in connection with the embodiment is included in at least one embodiment of the disclosure. The appearances of the phrase “in one embodiment” or “in one aspect” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

Provided herein are methods of predicting rates of progression of AMD and determining the presence of reticular pseudodrusen (RPD) using color fundus photography (CFP). AMD is classified into early, intermediate, and late stages. Late AMD, the stage associated with severe visual loss, occurs in two forms, geographic atrophy (GA) and neovascular AMD (NV). Making accurate time-based predictions of progression to late AMD is clinically critical. Making predictions of late stage AMD will enable improved decision-making regarding: (i) medical treatments, especially oral supplements known to decrease progression risk, (ii) lifestyle interventions, particularly smoking cessation and dietary changes, and (iii) intensity of patient monitoring, e.g., frequent reimaging in clinic and/or tailored home monitoring programs.

RPD, also known as subretinal drusenoid deposits, have been identified as a disease feature independently associated with increased risk of progression to late AMD. Unlike soft drusen, which are located in the sub-retinal pigment epithelial (RPE) space, RPD are thought to represent aggregations of material in the subretinal space between the RPE and photoreceptors. Compositional differences have also been found between soft drusen and RPD.

The detection of eyes with RPD is important for multiple reasons. Not only is their presence associated with increased risk of late AMD, but the increased risk is weighted towards particular forms of late AMD, including the recently recognized phenotype of geographic atrophy (GA) or also known as outer retinal atrophy (ORA). In recent analyses of Age-Related Eye Disease Study 2 (AREDS2) data, the risk of progression to GA was significantly higher with RPD presence, while the risk of neovascular AMD was not. Hence, RPD presence may be a powerfully discriminating feature that could be very useful in risk prediction algorithms for the detailed prognosis of AMD progression. The presence of RPD has also been associated with increased speed of GA enlargement, which is a key endpoint in ongoing clinical trials. Finally, in eyes with intermediate AMD, the presence of RPD appears to be a critical determinant of the efficacy of subthreshold nanosecond laser to slow progression to late AMD.

However, owing to the poor visibility of RPD on clinical examination and on color fundus photography (CFP), they were not incorporated into the traditional AMD classification and risk stratification systems, such as the Beckman clinical classification scale or the AREDS scales. With the advent of more recent imaging modalities, including fundus autofluorescence (FAF), near-infrared reflectance (NIR), and optical coherence tomography (OCT), the presence of RPD may be ascertained more accurately with careful grading at the reading center level. However, their detection by ophthalmologists (including retinal specialists) in a clinical setting may still be challenging.

CFP is the most widespread and accessible retinal imaging modality used worldwide; it is the most highly validated imaging modality for AMD classification and prediction of progression to late disease. Currently, two existing standards are available clinically for using CFP to predict the risk of progression of AMD. Simplified Severity Scale (SSS) is a points-based system whereby an examining physician scores the presence of two AMD features (macular drusen and pigmentary abnormalities) in both eyes of an individual. From the total score of 0-4, a five-year risk of late AMD is then estimated. The other standard is an online risk calculator. Like the SSS, its inputs include the presence of macular drusen and pigmentary abnormalities; however, it can also receive the individual's age, smoking status, and basic genotype information consisting of two SNPs (when available). Unlike the SSS system, the online risk calculator predicts the risk of progression to late AMD, GA, and NV at 1-10 years.

Both existing clinical standards face limitations. First, the ascertainment of the SSS features from CFP or clinical examination requires significant clinical expertise, typical in retinal specialists, but remains time-consuming and error-prone, even when performed by expert graders in a reading center. Second, the SSS relies on two hand-crafted features and cannot receive other potentially risk-determining features. Recent work applying deep learning (DL) has shown promise in the automated diagnosis and triage of conditions including cardiac, pediatric, dermatological, and retinal diseases, but not in predicting the risk of AMD progression on a large scale or at the patient level. However, this approach relied on previously published 5-year risk estimates at the severity class level, rather than using the ground truth of actual progression/non-progression at the level of individual eyes, or the timing and subtype of any progression events. In addition, no external validation using an independent dataset was performed in that study.

Deep learning is a branch of machine learning that allows computers to learn by example; in the case of image analysis, it involves training algorithmic models on images with accompanying labels such that they can perform classification of novel images according to the same labels. The models are typically neural networks that are constructed of an input layer (which receives the image), followed by multiple layers of non-linear transformations, to produce a classifier output (e.g., drusen and pigmentary abnormalities, RPD present or absent, etc.).

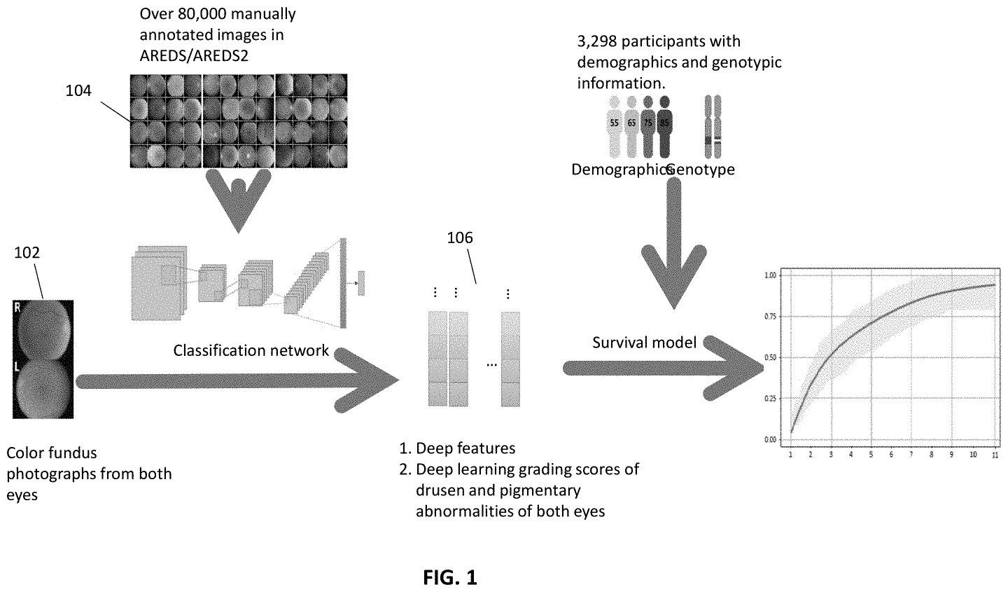

In some embodiments, the method of predicting the rate of progression of AMD provided herein may include a deep learning (DL) architecture to predict progression of AMD with improved accuracy and transparency in two steps: image classification followed by survival analysis, as seen in FIG. 1.

In some aspects, the prediction method performs progression predictions directly from CFP over a wide time interval (1-12 years). Second, training and testing may be based on the ground truth of reading center-graded progression events at the level of individuals. Both training and testing may utilize an expanded dataset with many more progression events. Third, the prediction method may predict the risk not only of late AMD, but also of GA and NV separately. This is important since treatment approaches for the two subtypes of late AMD are very different: NV needs to be diagnosed extremely promptly, since delay in access to intravitreal anti-VEGF injections is usually associated with very poor visual outcomes, while various therapeutic options to slow GA enlargement are under investigation. In addition, the two-step approach has important advantages. By separating the DL extraction of retinal features from the survival analysis, the final predictions are more explainable and biologically plausible, and error analysis is possible. By contrast, end-to-end ‘black-box’ DL approaches are less transparent and may be more susceptible to failure. The prediction method delivers autonomous predictions of a higher accuracy than those from retinal specialists using two existing clinical standards. Hence, the predictions are closer to the ground truth of actual time-based progression to late AMD than when retinal specialists are grading the same bilateral CFP and entering these grades into the SSS or the online calculator. In addition, deep feature extraction may generally achieve higher accuracy than DL grading of traditional hand-crafted features.

In addition, unlike the SSS, whose five-year risk prediction becomes saturated at 50%, the DL prediction methods herein enable ascertainment of risk above 50%. This may be helpful in justifying medical and lifestyle interventions, vigilant home monitoring, and frequent reimaging, and in planning shorter but highly powered clinical trials. For example, the AREDS-style oral supplements decrease the risk of developing late AMD by approximately 25%, but only in individuals at higher risk of disease progression. Similarly, if subthreshold nanosecond laser treatment is approved to slow progression to late AMD, accurate risk predictions may be very helpful for identifying eyes that may benefit most.

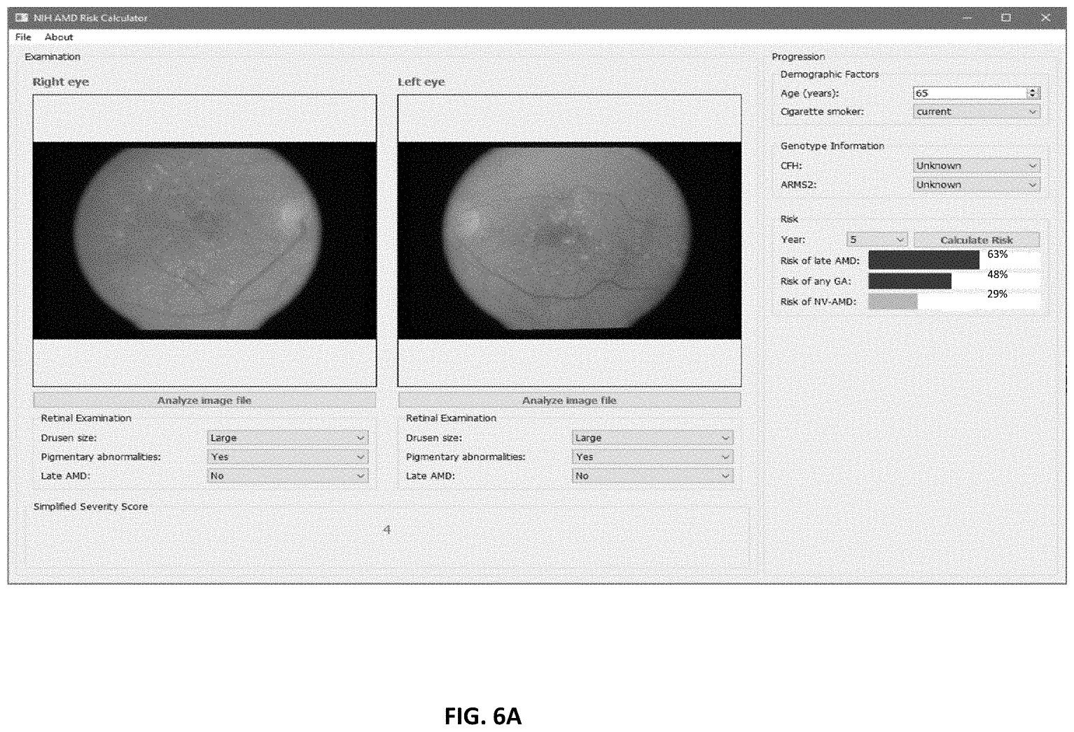

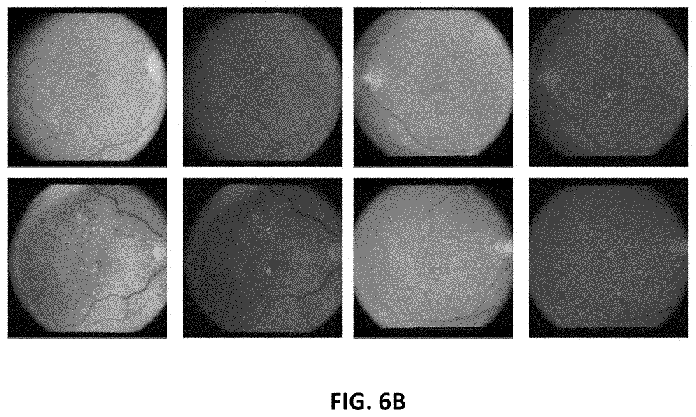

The overall framework of the prediction method is shown in FIG. 1. An example interface for the method is shown in FIG. 6A. At step 102, the prediction method may include receiving one or more CFP images from one or more eyes of a patient. In some examples, one or more CFP images from both eyes of the patient are received. At steps 104 and 106, a deep neural network (DNN) such as a deep convolutional neural network (CNN) may be adapted to classify each CFP image by either: (i) extracting multiple highly discriminative deep features, and/or (ii) estimating grades for drusen and pigmentary abnormalities. In some examples, the deep features may include drusen and/or pigmentary abnormalities. In this example, the drusen may be soft, large drusen.

In some examples, the classification of each CFP image may be performed using one or more adaptations of ‘DeepSeeNet’. DeepSeeNet is a CNN framework that was created for AMD severity classification. It has achieved state-of-the-art performance for the automated diagnosis and classification of AMD severity from CFP; this includes the grading of macular drusen, pigmentary abnormalities, the SSS, and the AREDS 9-step severity scale. For example, based on the received images, the following information may be automatically generated separately for each eye: drusen size status, pigmentary abnormality presence/absence, late AMD presence/absence, and the Simplified Severity Scale score.

In an example, the CNN may include embedded denoising operators for improved robustness. The denoising operators in the CNN may defend against unnoticeable adversarial attacks.

The method at step 106 may include extracting one or more ‘deep features’. In some examples, this step may involve using DL to derive and weight predictive image features, including high-dimensional ‘hidden’ features. Deep features may be extracted from the second to last fully-connected layer of DeepSeeNet (the highlighted portions in the classification network in FIG. 1). In total, 512 deep features may be extracted for each patient in this way, comprising 128 deep features for each model, drusen and pigmentary abnormalities, in each of the two images (left and right eyes). After feature extraction, all 512 deep features may be normalized as standard-scores.

In some examples, the method may further include feature selection to avoid overfitting and to improve the generalizability, because of the multi-dimensional nature of the features. Hence, performed selection may be performed to group correlated features and one feature may be picked for each group. Features with non-zero coefficients may be selected and applied as input to the survival models described below.

The method at step 106 may optionally include a second adaptation of DeepSeeNet (‘DL grading’). In this example, the method may include grading of drusen and pigmentary abnormalities, the two macular features considered by humans most able to predict progression to late AMD. In this adaptation, the two predicted risk factors may be used directly. One CNN may be provided, where the CNN has been previously trained and validated to estimate drusen status in a single CFP, according to three levels (none/small, medium, or large), using reading center grades as the ground truth. A second CNN may be provided, where the second CNN has been previously trained and validated to predict the presence or absence of pigmentary abnormalities in a single CFP.

The probability of progression to late AMD (in either eye) may be automatically calculated, along with separate probabilities of geographic atrophy and neovascular AMD. For example, at step 108, the method may further include generating a survival model by using a Cox proportional hazards model to predict probability of progression to late AMD (and GA/NV, separately), based on the deep features (‘deep features/survival’) or the DL grading (‘DL grading/survival’). The method may further include optional step 110 in which additional participant information may be added to the survival model, such as demographic and (if available) genotype information, along with a time point for prediction. For example, the patient's age, smoking status, and/or genetics may be received. In some examples, the probability of progression to late AMD may estimate time to late AMD. In some examples, the Cox model may be used to evaluate simultaneously the effect of several factors on the probability of the event, i.e., participant progression to late AMD in either eye. Separate Cox proportional hazards models may analyze time to late AMD and time to subtype of late AMD (i.e., GA and NV). In addition to the image-based information, the survival models may receive three additional inputs at step 110: (i) participant age; (ii) smoking status (current/former/never), and (iii) participant AMD genotype (CFH rs1061170, ARMS2 rs10490924, and the AMD GRS).

Also provided herein are methods of detecting RPD from one or more images of a patient's eye. In some examples, the one or more images may be CFP images, FAF images, ad/or near infrared images. In some examples, the method of detecting RPD in CFP images may be performed in conjunction with the method of predicting the risk of late AMD in a patient. For example, RPD may be detected in one or more CFP images of a patient and may be included as a deep feature for input into the survival model.

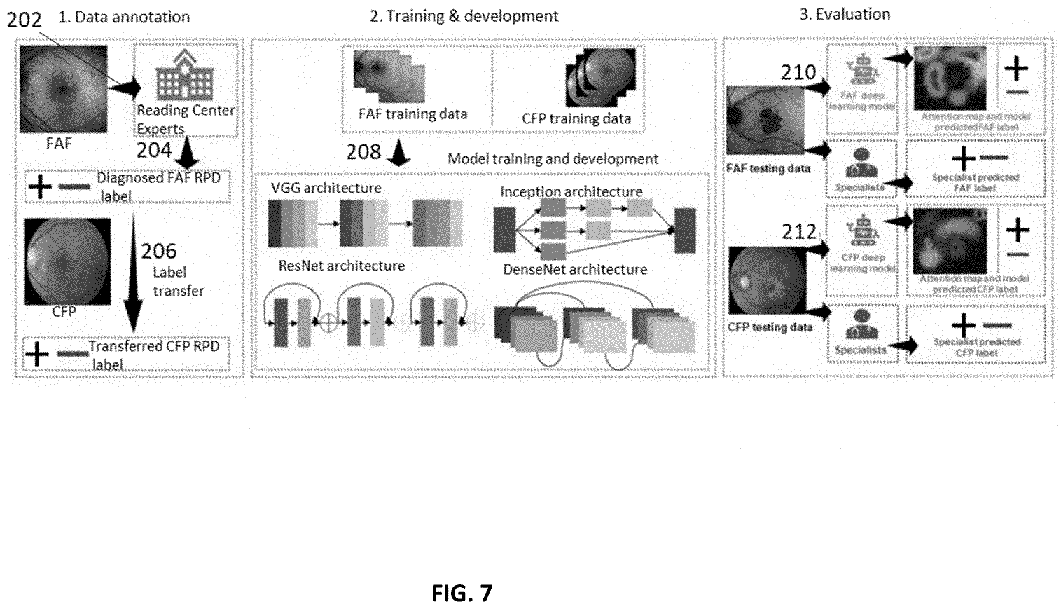

FIG. 7 is an overview of training a deep learning method for detecting RPD. At step 202, reading center experts may grade the presence or absence of reticular pseudodrusen (RPD) on fundus autofluorescence (FAF) images. At step 204, each FAF image may be assigned a label (e.g. RPD present or absent). These labels may be transferred to the CFP images at step 206. Then, at step 208, the FAF and CFP images may be each split into training, development, and test sets. In at least one example, one or more, such as ten deep learning models may be trained, about half for the FAF detection task and about half for the CFP detection task. Each model was evaluated on the hold-out test sets (steps 210 and 212). One or more (e.g. four) ophthalmologists also may grade a subset of the test sets. Their grades may be compared with those of the deep learning models, using the reading center grades as the ground truth.

In some examples, RPD may be detected in FAF images, regardless of image quality (e.g. high quality or low quality images). The method may detect RPD with higher accuracy than physicians or other trained specialists. The AUC may be high at about 0.94, driven principally by high specificity of about 0.97 (and lower sensitivity of about 0.70).

In some examples, the method of detecting RPD may include using label transfer from graded corresponding FAF images to serve as a standard for CFP images. In some examples, the method may identify RPD presence from CFP images with an AUC of about 0.83, including a relatively high specificity of about 0.90.

The ability to perform accurate detection of RPD presence in an efficient and accessible manner is of practical importance for multiple reasons. It enables a more detailed AMD classification and phenotyping that provides important diagnostic and prognostic information relevant to medical decision-making. Accurate RPD ascertainment can lead to more precise patient risk stratification for late AMD that can guide the institution of risk-reduction treatments (e.g., AREDS supplementation) and appropriate patient follow-up frequency. Given that RPD presence increases the likelihood of progression to late AMD, and preferentially to specific forms of late AMD, its inclusion in progression prediction methods can enable more accurate overall and subtype-specific predictions. Similarly, predictions of GA enlargement rates are likely to be improved, which would be important for patient recruitment, stratification, and data analysis in future clinical trials for GA intervention. Another example is experimental subthreshold nanosecond laser treatment of intermediate AMD, where RPD presence highly influences the effect of treatment on decreasing or conversely increasing progression risk to late AMD.

Using automated methods to ascertain RPD presence also has advantages over ascertainment by human graders/clinicians. Deep learning models can perform grading of large numbers of images very quickly, with complete consistency, and provide a confidence value associated with each binary prediction. Attention maps can be generated for each graded image to verify that the models are behaving with face validity. By contrast, human grading of RPD presence, particularly from CFP, is difficult to perform accurately, even by trained graders at the reading center level. This form of grading is operationally time-consuming and associated with lower rates of inter-grader agreement, relative to other AMD-associated features. Human grading of RPD presence from FAF images has higher accuracy than from CFP imaging, but additional FAF imaging in clinical care involves added time and expense, and grading expertise for FAF images is currently limited to a small number of specialist centers in developed countries.

With label transfer, deep learning models may be capable of ascertaining RPD presence from CFP images. This capability may unlock an additional dimension in CFP image datasets from established historical studies of AMD that are unaccompanied by corresponding images captured on other modalities and impractical to replicate with multimodal imaging. These include the AREDS, Beaver Dam Eye Study, Blue Mountains Eye study, and other population-based and AMD-based longitudinal datasets, which have provided a wealth of information on AMD epidemiology and genetics.

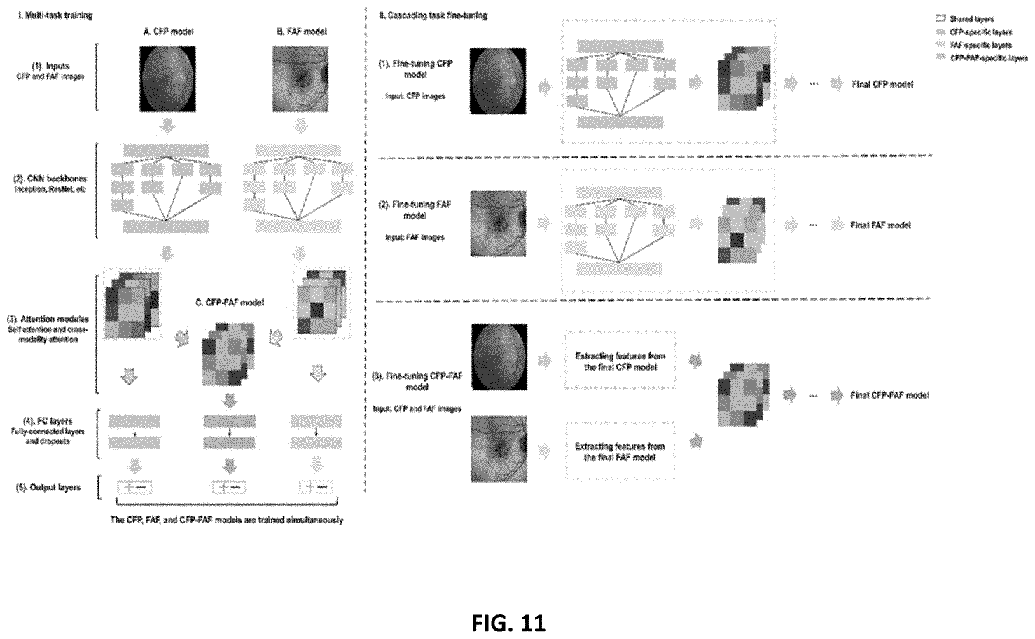

FIG. 11 shows deep learning framework for an example, multi-modal, multi-task nature of training, and multi-attention mechanism (M3). The framework consists of three deep learning models: the CFP model, the FAF model, and the CFP-FAF model. The CFP model takes CFP images as its input and predicts RPD presence/absence as its output; the same idea applies to the FAF and CFP-FAF models. For the CFP model and FAF model, each has a CNN to extract features from the input image, followed by an attention module to analyze the features that contribute most to decision-making, followed by fully-connected layers, and an output layer, which makes the prediction. The CFP-FAF model has the same structure except that, instead of having its own CNN backbone, it receives the image features from both the CFP and the FAF models, Multi-task training may be used to train the deep learning models. As shown in FIG. 11, this may include (i) multi-task learning and (ii) cascading task fine-tuning. In multi-task learning, the models may be trained jointly, with each model considered as a parallel task, using a shared representation. In cascading task fine-tuning, each model then undergoes additional training separately. The aim of multi-task learning is to learn generalizable and shared representations for all the image scenarios, and the aim of cascading task fine-tuning is to perform additional training suitable for each separate image scenario.

Multi-task training has important advantages over traditional single-task learning, where each model is trained separately. Single-task training has the disadvantage that the performance of each model is limited by the features present on that particular image modality. Models trained in this way may also be more susceptible to overfitting. By contrast, multi-task training exploits the similarities (shared image features) and differences (task-specific image features) between the features present on the different image modalities. In this way, it usually has improved learning efficiency and accuracy. Essentially, what is learned for each image modality task can assist during training for the other image modality tasks. In this way, it benefits each model by sharing features that are generalizable between the image modalities. This may be particularly relevant for retinal lesions like RPD, where different imaging modalities (CFP and FAF) highlight very different features relating to the same underlying anatomy.

In addition, many existing multi-modality deep learning models simply concatenate features from each image modality. However, CFP and FAF are very different modalities and they have substantially different features. To address this problem, self-attention and cross-modality attention modules were employed, combined with the multi-task training (FIG. 11). For the CFP and FAF models, the self-attention module was used to find the most important features extracted from the CNN backbones. Then, the cross-modality attention module was used to combine the features learnt from the self-attention modules. Importantly, the two self-attention modules (from each of the two image modalities) are shared between all three models.

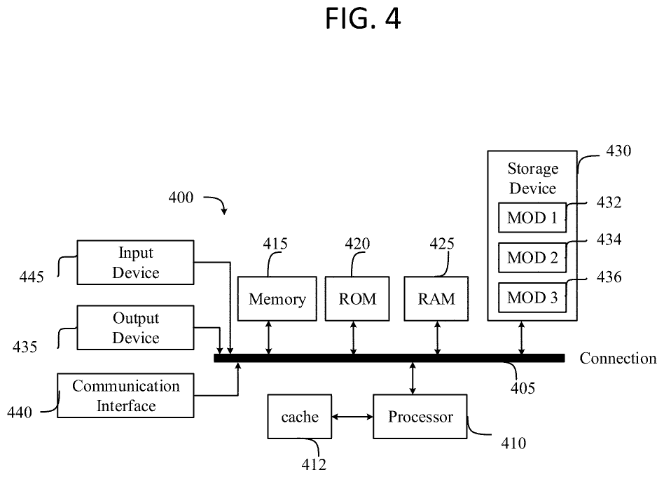

The disclosure now turns to the example system illustrated in FIG. 4 which may be used to implement the methods for predicting risk of late AMD and/or detecting RPD. FIG. 4 shows an example of computing system 400 in which the components of the system are in communication with each other using connection 405. Connection 405 can be a physical connection via a bus, or a direct connection into processor 410, such as in a chipset or system-on-chip architecture. Connection 405 can also be a virtual connection, networked connection, or logical connection.

In some examples computing system 400 is a distributed system in which the functions described in this disclosure can be distributed within a datacenter, multiple datacenters, a peer network, throughout layers of a fog network, etc. In some examples, one or more of the described system components represents many such components each performing some or all of the function for which the component is described. In some examples, the components can be physical or virtual devices.

Example system 400 includes at least one processing unit (CPU or processor) 410 and connection 405 that couples various system components including system memory 415, read only memory (ROM) 420 or random access memory (RAM) 425 to processor 410. Computing system 400 can include a cache of high-speed memory 412 connected directly with, in close proximity to, or integrated as part of processor 410.

Processor 410 can include any general purpose processor and a hardware service or software service, such as services 432, 434, and 436 stored in storage device 430, configured to control processor 410 as well as a special-purpose processor where software instructions are incorporated into the actual processor design. Processor 410 may essentially be a completely self-contained computing system, containing multiple cores or processors, a bus, memory controller, cache, etc. A multi-core processor may be symmetric or asymmetric.

To enable user interaction, computing system 400 includes an input device 445, which can represent any number of input mechanisms, such as a microphone for speech, a touch-sensitive screen for gesture or graphical input, keyboard, mouse, motion input, speech, etc. Computing system 400 can also include output device 435, which can be one or more of a number of output mechanisms known to those of skill in the art. In some instances, multimodal systems can enable a user to provide multiple types of input/output to communicate with computing system 400. Computing system 400 can include communications interface 440, which can generally govern and manage the user input and system output, and also connect computing system 400 to other nodes in a network. There is no restriction on operating on any particular hardware arrangement and therefore the basic features here may easily be substituted for improved hardware or firmware arrangements as they are developed.

Storage device 430 can be a non-volatile memory device and can be a hard disk or other types of computer readable media which can store data that are accessible by a computer, such as magnetic cassettes, flash memory cards, solid state memory devices, digital versatile disks, cartridges, battery backed random access memories (RAMs), read only memory (ROM), and/or some combination of these devices.

The storage device 430 can include software services, servers, services, etc., that when the code that defines such software is executed by the processor 410, it causes the system to perform a function. In some examples, a hardware service that performs a particular function can include the software component stored in a computer-readable medium in connection with the necessary hardware components, such as processor 410, connection 405, output device 435, etc., to carry out the function.

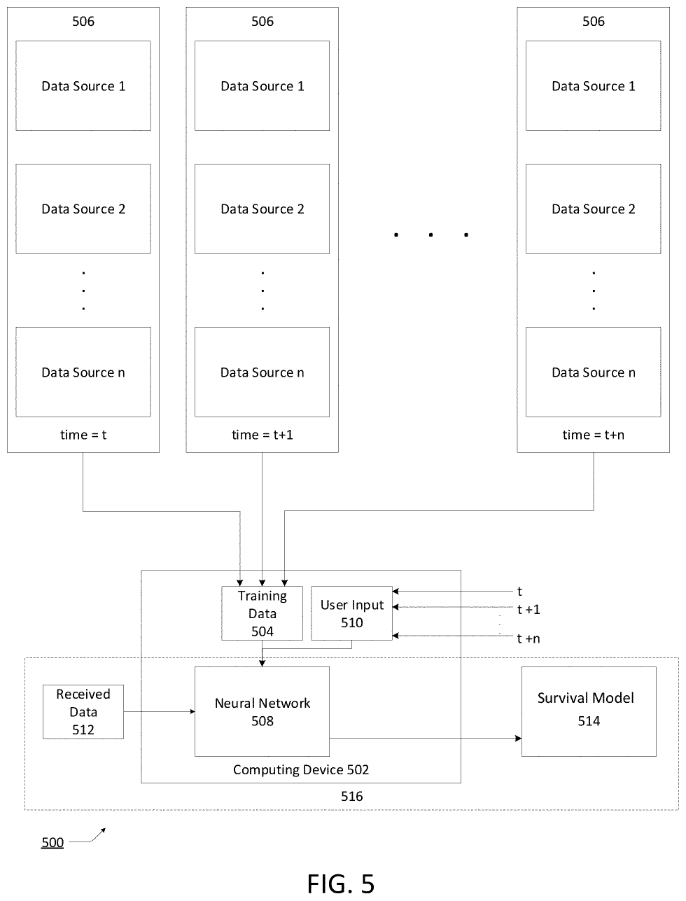

The disclosure now turns to FIG. 5, which illustrates an example machine learning environment 500. The machine learning environment can be implemented on one or more computing devices 502A-N (e.g., cloud computing servers, virtual services, distributed computing, one or more servers, etc.). The computing device(s) 502 can include training data 504 (e.g., one or more databases or data storage device, including cloud-based storage, storage networks, local storage, etc.). In some examples, the training data may include data from AREDS and/or AREDS2. The training data 504 of the computing device 502 can be populated by one or more data sources 506 (e.g., data source 1, data source 2, data source n, etc.) over a period of time (e.g., t, t+1, t+n, etc.). In some examples, training data 504 can be labeled data (e.g., one or more tags associated with the data). For example, training data can be one or more images and a label (e.g., drusen status/size (none/small, medium, or large), the presence or absence of pigmentary abnormalities, and/or the presence or absence of RPD) can be associated with each image. The computing device(s) 502 can continue to receive data from the one or more data sources 506 until the neural network 508 (e.g., convolution neural networks, deep convolution neural networks, artificial neural networks, learning algorithms, etc.) of the computing device(s) 502 are trained (e.g., have had sufficient unbiased data to respond to new incoming data requests and provided an autonomous or near autonomous image classification). In some examples, the neural network can be a convolutional neural network, for example, utilizing five layer blocks, including convolutional blocks, convolutional layers, and fully connected layers (e.g. ‘DeepSeeNet’, Densenet, Resnet, Inception v3, vgg16, or vgg19). While example neural networks are realized, neural network 508 can be one or more neural networks of various types are not specifically limited to a single type of neural network or learning algorithm.

In other examples, a feature selection can be generated (e.g., group correlated features such that one feature is used for each group). In these instances, features with non-zero coefficients are used in a survival model. The training data can require an equivalent number of images per patient, and as such, if a missing image exists a substitute image can be generated based on the existing images (e.g., in order to enable sufficient training data, while not biasing the training data).

In some examples, while not shown here, the training data 504 can be checked for biases, for example, by checking the data source 506 (and corresponding user input) verse previously known unbiased data. Other techniques for checking data biases are also realized. The data sources can be any of the sources of data for providing the input images (e.g., CFP, FAF, etc.) as described above in this disclosure.

The computing device(s) 502 can receive user (e.g., physician) input 510 related to the data source. The user input 510 and the data source 506 can be temporally related (e.g., by time t, t+1, t+n, etc.). That is, the user input 510 and the data sources 506 can be synchronous in that the user input 510 corresponds and supplements the data source 506 in a manner of supervised or reinforced learning. For example, a data source 506 can provide a CFP and/or FAF image at time t and corresponding user input 510 can be input of drusen size, RPD presence, and/or pigmentary abnormalities of that CFP and/or FAF image at time t. While, time t may actually be different in real-world time, they are synchronized in time with respect to the data provided to the training data.

The training data 504 can be used to train a neural network 508 or learning algorithms (e.g., convolutional neural network, artificial neural network, etc.). The neural network 508 can be trained, over a period of time, to automatically (e.g., autonomously) determine what the user input 510 would be, based only on received data 512 (e.g., imaging data, etc.). For example, by receiving a plurality of unbiased data and/or corresponding user input for a long enough period of time, the neural network will then be able to determine what the user input would be when provided with only the data. For example, a trained neural network 508 will be able to receive a CFP and/or FAF image (e.g., 512) and based on the CFP and/or FAF image determine the drusen size, RPD presence, and/or pigmentary abnormalities features or grading that a physician would manually identify (and that would have been provided as user input 510 during training). In some examples, this can be based on labels associated with the data as described above. The output from the trained neural network can be provided to a survival model 514 for treating a patient. In some examples, the output from the trained neural network can be inputted directly into a survival model to predict a rate of progression of late AMD in the patient.

Trained neural network system 516 can include a trained neural network 508, received data 512, and survival model 514. The received data 512 can be information related to a patient, as previously described above. The received data 512 can be used as input to trained neural network 508. Trained neural network 508 can then, based on the received data 512, label the received data and/or determine a recommended course of action for treating the patient, based on how the neural network was trained (as described above). The recommended course of action or output of trained neural network 508 can be used as an input into a survival model 514 (e.g., to predict the risk of progression to late AMD for the patient to which the received data 512 corresponds). In other instances, the output from the trained neural network can be provided in a human readable form, for example, to be reviewed by a physician to determine a course of action.

For clarity of explanation, in some instances the present technology may be presented as including individual functional blocks including functional blocks comprising devices, device components, steps or routines in a method embodied in software, or combinations of hardware and software.

In some embodiments the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer readable media. Such instructions can comprise, for example, instructions and data which cause or otherwise configure a general purpose computer, special purpose computer, or special purpose processing device to perform a certain function or group of functions. Portions of computer resources used can be accessible over a network. The computer executable instructions may be, for example, binaries, intermediate format instructions such as assembly language, firmware, or source code. Examples of computer-readable media that may be used to store instructions, information used, and/or information created during methods according to described examples include magnetic or optical disks, flash memory, USB devices provided with non-volatile memory, networked storage devices, and so on.

Devices implementing methods according to these disclosures can comprise hardware, firmware and/or software, and can take any of a variety of form factors. Typical examples of such form factors include laptops, smart phones, small form factor personal computers, personal digital assistants, rackmount devices, standalone devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

### EXAMPLES

### Example 1: Datasets

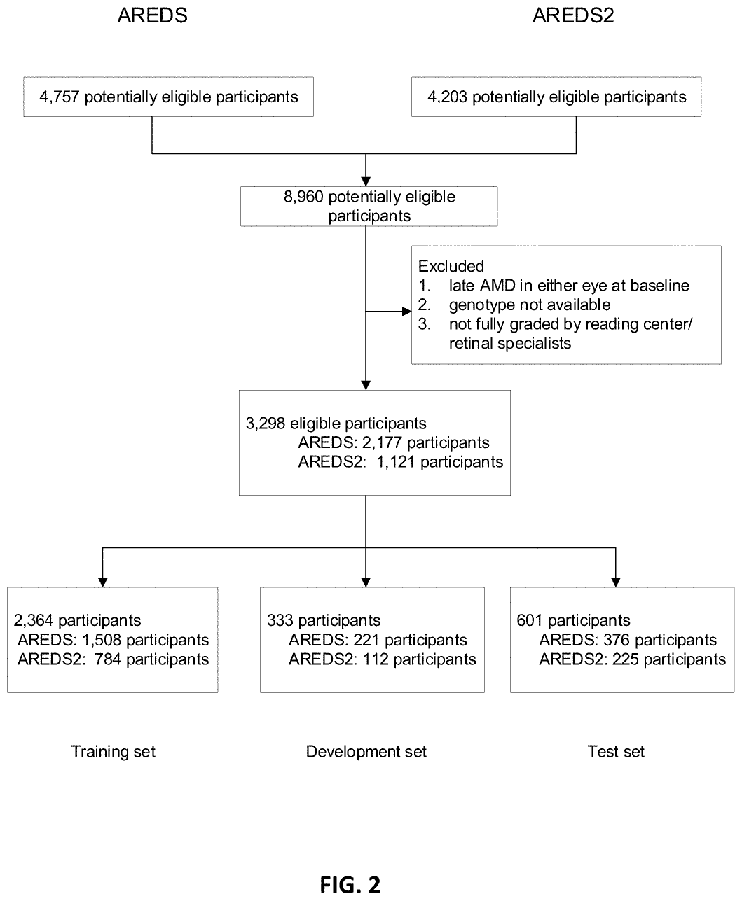

For model development and clinical validation of the methods of predicting risk of late AMD, two datasets were used: the AREDS and the AREDS2 (FIG. 2). For development of the methods of detecting RPD, the AREDS2 dataset was used. The AREDS was a 12-year multi-center prospective cohort study of the clinical course, prognosis, and risk factors of AMD, as well as a phase III randomized clinical trial (RCT) to assess the effects of nutritional supplements on AMD progression. In short, 4,757 participants aged 55 to 80 years were recruited between 1992 and 1998 at 11 retinal specialty clinics in the United States. The inclusion criteria were wide, from no AMD in either eye to late AMD in one eye. The participants were randomly assigned to placebo, antioxidants, zinc, or the combination of antioxidants and zinc. The AREDS dataset is publicly accessible to researchers by request at dbGAP.

Similarly, the AREDS2 was a multi-center phase III RCT that analyzed the effects of different nutritional supplements on the course of AMD. 4,203 participants aged 50 to 85 years were recruited between 2006 and 2008 at 82 retinal specialty clinics in the United States. The inclusion criteria were the presence of either bilateral large drusen or late AMD in one eye and large drusen in the fellow eye. The participants were randomly assigned to placebo, lutein/zeaxanthin, docosahexaenoic acid (DHA) plus eicosapentaenoic acid (EPA), or the combination of lutein/zeaxanthin and DHA plus EPA. AREDS supplements were also administered to all AREDS2 participants, because they were by then considered the standard of care.

In both studies, the primary outcome measure was the development of late AMD, defined as neovascular AMD or central GA. Institutional review board approval was obtained at each clinical site and written informed consent for the research was obtained from all study participants. The research was conducted under the Declaration of Helsinki and, for the AREDS2, complied with the Health Insurance Portability and Accessibility Act. For both studies, at baseline and annual study visits, comprehensive eye examinations were performed by certified study personnel using a standardized protocol, and CFP (field 2, i.e., 30° imaging field centered at the fovea) were captured by certified technicians using a standardized imaging protocol. Progression to late AMD was defined by the study protocol based on the grading of CFP, as described below.

In addition, the AREDS2 ancillary study of FAF imaging was conducted at 66 selected clinic sites, according to the availability of imaging equipment. Sites were permitted to join the ancillary study at any time after FAF imaging equipment became available during the five-year study period. Hence, while some sites performed FAF imaging on their participants from the AREDS2 baseline visit onwards, other sites performed FAF imaging from later study visits onwards, and the remaining sites did not perform FAF imaging at any point. The FAF images were acquired from the Heidelberg Retinal Angiograph (Heidelberg Engineering, Heidelberg, Germany) and fundus cameras with autofluorescence capability by certified technicians using standard imaging protocols. For the Heidelberg images, a single image was acquired at 30 degrees centered on the macula, captured in high speed mode (768×768 pixels), using the automated real time mean function set at 14. All images were sent to the University of Wisconsin Fundus Photographic Reading Center.

The ground truth labels used for both training and testing were the grades previously assigned to each FAF image by expert human graders at the University of Wisconsin Fundus Photograph Reading Center. The expert human grading team comprised six graders: four primary graders and two senior graders as adjudicators. The graders were certified technicians with over 10 years of experience in the detailed evaluation of retinal images for AMD. (These graders did not overlap at all with the four ophthalmologists described below). In short, RPD were defined as clusters of discrete round or oval lesions of hypoautofluorescence, usually similar in size, or confluent ribbon-like patterns with intervening areas of normal or increased autofluorescence; a minimum of 0.5 disc area (approximately five lesions) was required. Two primary graders at the reading center independently evaluated FAF images (from both initial and subsequent study visits) for the presence of RPD. In the case of disagreement between the two primary graders, a senior grader at the reading center would adjudicate the final grade. Inter-grader agreement for the presence/absence of RPD was 94%. Label transfer was used between the FAF images and their corresponding CFP images; this means that the ground truth label obtained from the reading center for each FAF image (RPD present or absent) was also applied to the corresponding CFP image (i.e., FAF-derived label, irrespective of the reading center grade for the CFP image itself). Separately, all of the corresponding CFP images were independently graded at the reading center for RPD, defined on CFP as an ill-defined network of broad interlacing ribbons. However, these grades were not used as the ground truth.

All available AREDS2 FAF images were used, i.e., including those from all visits. The image datasets were split randomly into three sets: 70% for training, 10% for validation, and 20% for testing of the models. The split was made at the participant level, such that all images from a single participant were present in only one of the three sets. The details of the datasets and splits are shown in Table 1, including the demographic characteristics of the study participants and the distribution of AMD severity levels of the images.

TABLE 1

| | Training | Validation | Test | |
|---|---|---|---|---|
| | set | set | set | Total |
| Participants (n) | 1,719 | 236 | 488 | 2,443 |
| Female sex (%) | 57.7 | 57.6 | 56.8 | 57.5 |
| Mean age (years) | 72.7 | 73.0 | 73.4 | 72.9 |
| Images (n)* | 7,930 | 1,096 | 2,249 | 11,275 |
| RPD present (%), gold-standard | 27.6 | 24.2 | 25.8 | 26.9 |
| label, as determined by reading | | | | |
| center grading of FAF images | | | | |
| AREDS 9-step AMD severity | | | | |
| scale distribution (%) | | | | |
| Steps 1-5 | 7.7 | 10.0 | 7.7 | 7.9 |
| Steps 6-8 | 53.0 | 52.0 | 50.6 | 52.4 |
| Geographic atrophy present | 14.1 | 14.1 | 16.0 | 14.5 |
| Neovascular AMD present | 25.1 | 23.5 | 25.4 | 25.0 |
| Not gradable | 0.2 | 0.4 | 0.3 | 0.2 |

*These refer to the total number of fundus autofluorescence and corresponding color fundus photograph image pairs, i.e., 11,275 fundus autofluorescence images and the 11,275 corresponding color fundus photographs that were captured on the same eye at the same study visit.

The total number of images in all three datasets was 11,275. Of the 4,724 study eyes, approximately half (51.5%) contributed only one image to the dataset: 51.4% (training set), 49.7% (validation set), and 52.6% (test set); the remaining eyes contributed more than one image. Overall, the mean number of images used per eye was 2.39: 2.39 (training set), 2.38 (validation set), and 2.39 (test set). The number of images where multiple images were used from the same eye was 8,983 images of 2,432 eyes: 6,316 images of 1,708 eyes (training set), 864 images of 229 eyes (validation set), and 1,803 images of 495 eyes (test set). Of these 8,893 images, the proportion with RPD was 27.1%: 27.9% (training set), 25.2% (validation set), and 25.0% (test set). Of the 738 eyes that contributed multiple images where at least one image had RPD, the proportion that had RPD from the first image used was 76.3%: 78.4% (training set), 71.2% (validation set), and 70.6% (test set).

As part of the studies, 2,889 (AREDS) and 1,826 (AREDS2) participants consented to genotype analysis. SNPs were analyzed using a custom IIlumina HumanCoreExome array. For the current analysis, two SNPs (CFH rs1061170 and ARMS2 rs10490924, at the two loci with the highest attributable risk of late AMD), were selected, as these are the two SNPs available as input for the existing online calculator system. In addition, the AMD GRS was calculated for each participant according to methods known in the art. The GRS is a weighted risk score based on 52 independent variants at 34 loci identified in a large genome-wide association study as having significant associations with risk of late AMD. The online calculator cannot receive this detailed information.

The eligibility criteria for participant inclusion in the current analysis were: (i) absence of late AMD (defined as NV or any GA) at study baseline in either eye, since the predictions were made at the participant level, and (ii) presence of genetic information (in order to compare model performance with and without genetic information on exactly the same cohort of participants). Accordingly, the images used for the predictions were those from the study baselines only.

In the AREDS dataset of CFPs, information on image laterality (i.e., left or right eye) and field status (field 1, 2, or 3) were available from the Reading Center. However, these were not available in the AREDS2 dataset of CFPs. Therefore, two Inception-v3 models were trained, one for classifying laterality and the other for identifying field 2 images. Both models were first trained on the gold standard images from the AREDS and fine-tuned on a newly created gold standard AREDS2 set manually graded by a retinal specialist (TK). The AREDS2 gold standard consisted of 40 participants with 5,164 images (4,097 for training and 1,067 for validation). The models achieved 100% accuracy for laterality classification and 97.9% accuracy (F1-score 0.971, precision 0.968, recall 0.973) for field 2 classification.

The ground truth labels used for both training and testing were the grades previously assigned to each CFP by expert human graders at the University of Wisconsin Fundus Photograph Reading Center. The RPD grading was performed from FAF images (since RPD are detected by human experts with far greater accuracy on FAF images than on CFP). The grading for geographic atrophy and pigmentary abnormalities was performed from CFP (since this remains the gold standard for grading pigmentary abnormalities and was traditionally considered the gold standard for grading geographic atrophy). In brief, the reading center workflow includes a senior grader performed initial grading of each photograph for AMD severity using a 4-step scale and a junior grader performed detailed grading for multiple AMD-specific features. All photographs were graded independently and without access to the clinical information. A rigorous process of grading quality control was performed at the reading center, including assessment for inter-grader and intra-grader agreement. The reading center grading features relevant to the current study, aside from late AMD, were: (i) macular drusen status (none/small, medium (diameter ≥63 μm and <125 μm), and large (≥125 μm)), and (ii) macular pigmentary abnormalities related to AMD (present or absent). RPD were defined as clusters of discrete round or oval lesions of hypoautofluorescence, usually similar in size, or confluent ribbon-like patterns with intervening areas of normal or increased autofluorescence; a minimum of 0.5 disc area (approximately five lesions) was required. Label transfer was used between the FAF images and their corresponding CFP images; this means that the ground truth label obtained from the reading center for each FAF image was also applied to the corresponding CFP. Similarly, the labels from the FAF images were also applied to the CFP-FAF image pairs.

In addition to undergoing reading center grading, the images at the study baseline were also assessed (separately and independently) by 88 retinal specialists in AREDS and 196 retinal specialists in AREDS2. The responses of the retinal specialists were used not as the ground truth, but for comparisons between human grading as performed in routine clinical practice and DL-based grading. By applying these retinal specialist grades as input to the two existing clinical standards for predicting progression to late AMD, it was possible to compare the current clinical standard of human predictions with those predictions achievable by DL.

### Example 2: Deep Learning Models

The convolutional neural network (CNN), a type of deep learning model designed for image classification, has become the state-of-the-art method for the automated identification of retinal diseases from CFP. For the disclosed methods, the CNN DenseNet (version 152) was used to create the classification network for AMD, the survival model, the FAF deep learning model, and the CFP deep learning model for the binary detection of RPD presence/absence. This new CNN contains 601 layers, comprising a total of over 14 million weights (learnable parameters) that are subject to training. Its main novel feature is that each layer passes on its output to all subsequent layers, such that every layer obtains inputs from all the preceding layers (not just the immediately preceding layer, as in previous CNNs). DenseNet has demonstrated superior performance to many other slightly older CNNs in a range of image classification applications. However, for comparison of performance according to the CNN used, we trained additional deep learning models using four different CNNs frequently employed in image classification tasks: VGG version 16, VGG version 19, InceptionV3, and ResNet version 101 (i.e., eight additional deep learning models in total).

All deep learning models were pre-trained using ImageNet, an image database of over 14 million natural images with corresponding labels, using methods described previously. (This very large dataset is often used in deep learning to pre-train models. Pre-training on ImageNet is used to initialize the layers/weights, leading to the recognition of primitive features (e.g., edge detection), prior to subsequent training on the dataset of interest). The models were trained with two commonly used libraries, Keras and TensorFlow. During the training process, each image was scaled to 512×512 pixels. Aside from this, no image preprocessing was performed for either the FAF or CFP images. The model parameters were updated using the Adam optimizer (learning rate of 0.0001) for every minibatch of 16 images. Model convergence was measured when the loss on the training set started to increase. The training was stopped five epochs (passes of the entire training set) after the loss of the training set no longer decreased. All experiments were conducted on a server with 32 Intel Xeon CPUs, using three NVIDIA GeForce GTX 1080 Ti 11 Gb GPUs for training and testing, with 512 Gb available in RAM memory. In addition, image augmentation procedures were used, as follows, in order to increase the dataset size and to strengthen model generalizability: (i) rotation (180 degrees), (ii) horizontal flip, and (iii) vertical flip.

In both of the DeepSeeNet adaptations described, the DL CNNs used Inception-v3 architecture, which is a state-of-the-art CNN for image classification; it contains 317 layers, comprising a total of over 21 million weights that are subject to training. Training was performed using two commonly used libraries: Keras and TensorFlow. All images were cropped to generate a square image field encompassing the macula and resized to 512×512 pixels. The hyperparameters were learning rate 0.0001 and batch size 32. The training was stopped after 5 epochs once the accuracy on the development set no longer increased. All experiments were conducted on a server with 32 Intel Xeon CPUs, using a NVIDIA GeForce GTX 1080 Ti 11 Gb GPU for training and testing, with 512 Gb available in RAM memory. We performed feature selection using the ‘glmnet’ package in R version 3.5.2 statistical software.

### Example 3: Robust Convolutional Neural Networks Against Adversarial Attacks

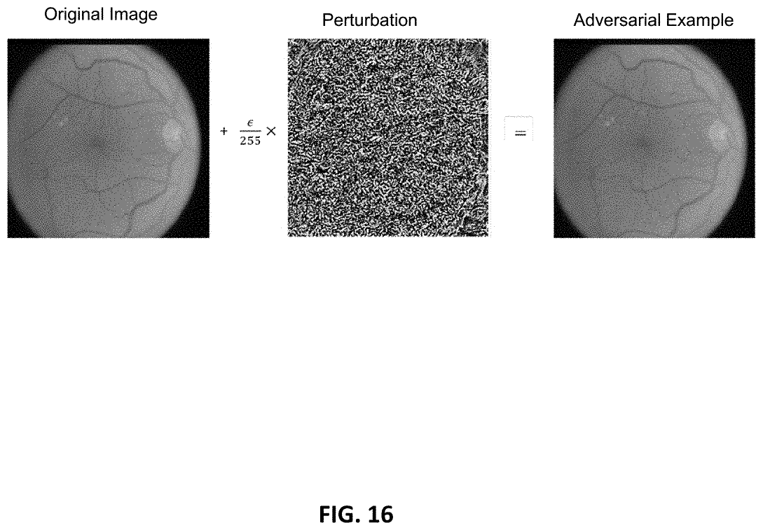

CNNs are vulnerable to adversarial attacks (FIG. 16). Images can be attacked by adding a small adversarial perturbation to the original images; the perturbation is imperceptible to humans but misleads a standard CNN model into producing incorrect outputs, with a substantial decline in its predictive performance. As a result, CNNs under adversarial attacks would fail to assist and might even mislead human clinicians. Importantly, such a vulnerability also poses severe security risks and represents a barrier to the deployment of automated CNN-based systems in real-world use, especially in the medical domain where accurate diagnostic results are of paramount importance in patient care.

It was hypothesized that noise (irrelevant or corrupted discriminative information) learned during CNN training is of considerable importance to CNNs' robustness against adversarial attacks. More specifically, images usually contain many noisy features that are irrelevant to human classification. CNNs are likely to learn these noisy features unconsciously during training, resulting in noise in feature representations for decision-making. As such, adversarial perturbations might manipulate the noise in feature representations to degrade model accuracy.

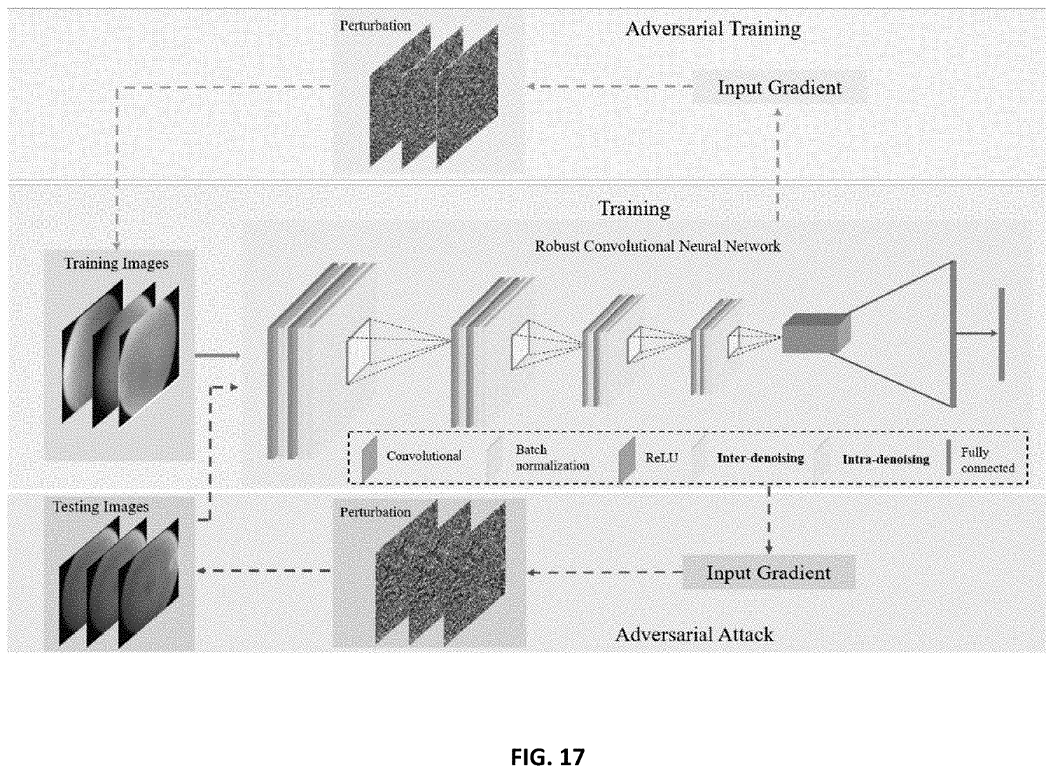

To alleviate the effect of noisy features learned during training, a robust CNN framework was developed (FIG. 17), in which a novel denoising operator was embedded into each convolutional layer to reduce the noise in its outputs, thereby combatting the effect of adversarial perturbations. The denoising operator contained two layers: an inter-sample denoising layer and an intra-sample denoising layer. The former utilized the entire batch of data to decrease the noise contained in feature representations, which might otherwise be mistaken under adversarial attacks as discriminative features. The latter reduced the noise in each medical image itself, to further lower the noise in feature representations.

The root causes of vulnerability in medical DL-systems were examined and a new defense method for improving their robustness against adversarial attacks was subsequently proposed. To illustrate and quantify the scale of adversarial perturbation imperceptible to humans, both radiologists and retinal specialists were first recruited to distinguish whether and to what degree attacked images can be distinguished from their original versions. To demonstrate the generalizability of this method, experiments were conducted on CFPs. Both white-box and black-box attack scenarios were taken into consideration and demonstrated the validity of the method under various state-of-the-art attacking methods. Black-box attacks are more realistic than white-box attacks.

In the two denoising layers, inter-sample denoising is the key to defend against both adversarial and transferable adversarial examples, probably because it utilizes the other samples to reduce the noise in feature representations of one sample in each batch. Meanwhile, intra-sample denoising can further enhance model robustness. However, inter-sample denoising might require more testing time, when images are tested one by one. This is because inter-sample denoising requires a different set of images to reduce the noise in feature representations of each testing image. The success of combining adversarial training and the method suggests that adversarial training might be complementary to the denoising layers in the method by further decreasing the noise manipulated by adversarial perturbations.

The experimental results suggest that, if CNNs can eliminate all noise in their learned feature representations, they would be more robust against adversarial perturbations.

### Example 4: Deep Learning Models Trained on the Combined AREDS/AREDS2 Training sets and validated on the combined AREDS/AREDS2 test sets

For training and testing the risk of late AMD prediction framework, both the AREDS and AREDS2 datasets were used. The characteristics of the participants are shown in Table 2. In the primary set of experiments, eligible participants from both studies were pooled to create one broad cohort of 3,298 individuals that combined a wide spectrum of baseline disease severity with a high number of progression events. The combined dataset was split at the participant level in the ratio 70%/10%/20% to create three sets: 2,364 participants (training set), 333 participants (development set), and 601 participants (hold-out test set).

TABLE 2

| Characteristics | AREDS | AREDS2 |
|---|---|---|
| Participants characteristics | | |
| Number of participants | 2,177 | 1,121 |
| Age, mean (SD), y | 68.4 (4.8) | 70.9 (7.9) |
| Smoking history (never, former, current), % | 49.6/45.6/4.8 | 45.9/48.5/5.5 |
| CFH rs1061170 (TT/CT/CC), % | 33.3/46.4/20.3 | 17.8/41.7/40.5 |
| ARMS2 rs10490924 (GG/GT/TT), % | 55.2/36.5/8.3 | 40.5/42.8/16.7 |
| AMD Genetic Risk Score, mean (SD) | 14.2 (1.4) | 15.2 (1.3) |
| Follow-up, Median (IQR), y | 10.0 (4.0) | 5.0 (1.0) |
| Progression to late AMD (classified by Reading Center) | | |
| Late AMD: % of participants at year 1/2/3/4/5/all years | 1.5/3.9/6.68/8.6/10.7/18.5 | 9.1/16.2/23.8/32.6/38.1/38.8 |
| GA: % of participants at year 1/2/3/4/5/all years | 0.6/1.5/2.8/4.0/5.1/10.1 | 4.8/9.0/13.0/17.8/20.8/21.0 |
| NV: % of participants at year 1/2/3/4/5/all years | 0.9/2.4/4.0/4.6/5.5/8.4 | 4.3/7.2/10.8/14.7/17.3/17.8 |

All of the baseline images in the test set were graded by 88 (AREDS) and 192 (AREDS2) retinal specialists. By using these grades as input to either the SSS or the online calculator, the prediction results of the two existing standards were computed: ‘retinal specialists/SSS’ and ‘retinal specialists/calculator’.

For three of the four approaches (deep features/survival, DL grading/survival, and retinal specialists/calculator), the input was bilateral CFP, participant age, and smoking status; separate experiments were conducted with and without the additional input of genotype data. For the other approach (retinal specialists/SSS), the input was bilateral CFP only.

In addition to the primary set of experiments where eligible participants from the AREDS and AREDS2 were combined to form one dataset, separate experiments were conducted where the DL models were: (i) trained separately on the AREDS training set only, or the AREDS2 training set only, and tested on the combined AREDS/AREDS2 test set, and (ii) trained on the AREDS training set only and externally validated by testing on the AREDS2 test set only.

The prediction accuracy of the approaches was compared using the five-year C-statistic as the primary outcome measure. The five-year C-statistic of the two DL approaches met and substantially exceeded that of both existing standards (Table 3). For predictions of progression to late AMD, the five-year C-statistic was 86.4 (95% confidence interval 86.2-86.6) for deep features/survival, 85.1 (85.0-85.3) for DL grading/survival, 82.0 (81.8-82.3) for retinal specialists/calculator, and 81.3 (81.1-81.5) for retinal specialists/SSS. For predictions of progression to GA, the equivalent results were 89.6 (89.4-89.8), 87.8 (87.6-88.0), and 82.6 (82.3-82.9), respectively; while these are not available for retinal specialists/SSS, since the SSS does not make separate predictions for GA or NV. For predictions of progression to NV, the equivalent results were 81.1 (80.8-81.4), 80.2 (79.8-80.5), and 80.0 (79.7-80.4), respectively.

TABLE 3

| Models | 1 | 2 | 3 | 4 | 5 | All years |
|---|---|---|---|---|---|---|
| Late AMD | | | | | | |
| Deep features/survival | 87.8(87.5, 88.1) | 85.8(85.4, 86.2) | 86.3(86.1, 86.6) | 86.7(86.5, 86.9) | 86.4(86.2, 86.6) | 86.7(86.5, 86.8) |
| DL grading/survival | 84.9(84.6, 85.3) | 84.1(83.8, 84.4) | 84.8(84.5, 85.0) | 84.8(84.6, 85.0) | 85.1(85.0, 85.3) | 84.9(84.6, 85.3) |
| Retinal | — | 78.3(77.9, 78.8) | 81.8(81.5, 82.1) | 82.7(82.4, 82.9) | 82.0(81.8, 82.3) | — |
| specialists/calculator | | | | | | |
| Retinal specialists/SSS* | — | — | — | — | 81.3(81.1, 81.5) | — |
| Geographic atrophy | | | | | | |
| Deep features/survival | 89.2(88.9, 89.6) | 91.0(90.7, 91.2) | 88.7(88.4, 88.9) | 89.1(88.9, 89.3) | 89.6(89.4, 89.8) | 89.2(89.1, 89.4) |
| DL grading/survival | 88.6(88.3, 88.9) | 86.6(86.4, 86.9) | 87.6(87.4, 87.9) | 88.1(87.9, 88.3) | 87.8(87.6, 88.0) | 88.6(88.3, 88.9) |
| Retinal | — | 77.5(76.9, 78.0) | 81.2(80.8, 81.6) | 82.0(81.6, 82.3) | 82.6(82.3, 82.9) | — |
| specialists/calculator | | | | | | |
| Retinal specialists/SSS* | — | — | — | — | — | — |
| Neovascular AMD | | | | | | |
| Deep features/survival | 85.4(84.9, 85.9) | 77.9(77.3, 78.5) | 81.7(81.3, 82.1) | 81.7(81.4, 82.1) | 81.1(80.8, 81.4) | 82.1(81.8, 82.4) |
| DL grading/survival | 78.0(77.4, 78.6) | 78.4(77.9, 78.9) | 79.2(78.8, 79.6) | 79.0(78.7, 79.4) | 80.2(79.8, 80.5) | 78.0(77.4, 78.6) |
| Retinal | — | 75.5(74.8, 76.2) | 81.7(81.2, 82.1) | 81.4(81.0, 81.8) | 80.0(79.7, 80.4) | — |
| specialists/calculator | | | | | | |
| Retinal specialists/SSS* | — | — | — | — | — | — |

*Retinal specialists/SSS
—makes predictions at one fixed interval of five years and for late AMD only (i.e., not by disease subtype); unlike all other models, for SSS, late AMD is defined as NV or central GA (instead of NV or any GA)

Similarly, for predictions at 1-4 years, the C-statistic was higher in all cases for the two DL approaches than the retinal specialists/calculator approach. Of the two DL approaches, the C-statistics of deep features/survival were higher in most cases than those of DL grading/survival. Predictions at these time intervals were not available for retinal specialists/SSS, since the SSS does not make predictions at any interval other than five years.

Regarding the separate predictions of progression to GA and NV, deep features/survival also provided the most accurate predictions at most time intervals. Overall, DL-based image analysis provided more accurate predictions than those from retinal specialist grading using the two existing standards. For deep feature extraction, this may reflect the fact that DL is unconstrained by current medical knowledge and not limited to two hand-crafted features.

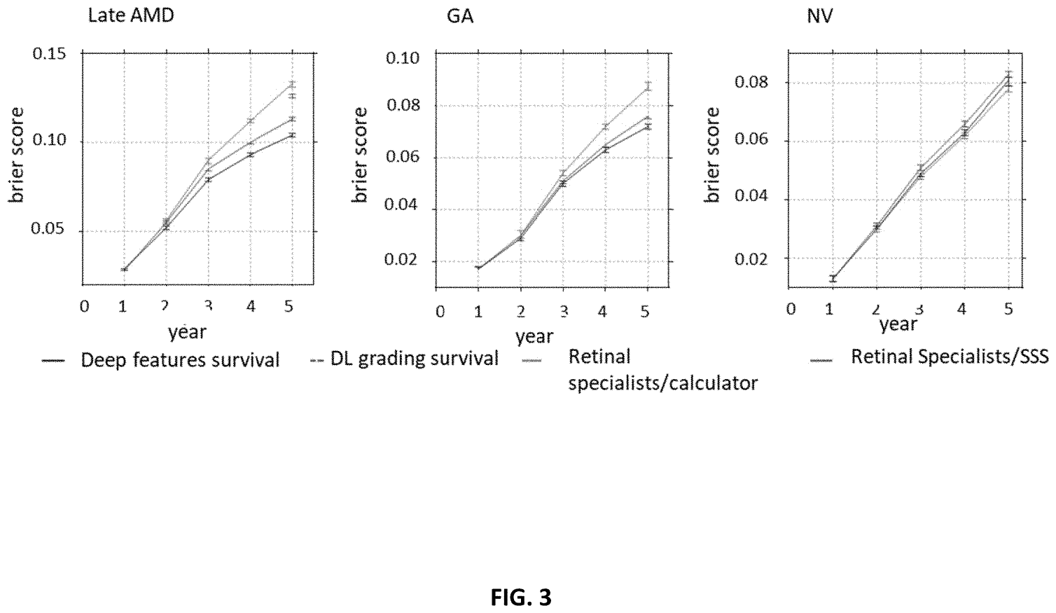

In addition, the prediction calibrations were compared using the Brier score (FIG. 3). For five-year predictions of late AMD, the Brier score was lowest (i.e., optimal) for deep features/survival.

Deep learning models were trained separately on individual cohorts (either AREDS or AREDS2) and validated on the combined AREDS/AREDS2 test sets. Models trained on the combined AREDS/AREDS2 cohort (Table 3) were substantially more accurate than those trained on either individual cohort (Table 4), with the additional advantage of improved generalizability. Indeed, one challenge of DL has been that generalizability to populations outside the training set can be variable. In this instance, the widely distributed sites and diverse clinical settings of AREDS/AREDS2 participants, together with the variety of CFP cameras used, help provide some assurance of broader generalizability.

TABLE 4

| | Trained on | Trained on |
|---|---|---|
| Models | AREDS | AREDS2 |
| Late AMD | | |
| Deep features/survival | 85.7(85.5, 85.9) | 83.9(83.7, 84.1) |
| DL grading/survival | 84.7(84.5, 84.9) | 82.1(81.8, 82.3) |
| Geographic atrophy | | |
| Deep features/survival | 89.3(89.1, 89.5) | 84.7(84.4, 85.0) |
| DL grading/survival | 90.2(90.0, 90.4) | 85.2(84.9, 85.5) |
| Neovascular AMD | | |
| Deep features/survival | 79.6(79.3, 80.0) | 74.0(73.6, 74.5) |
| DL grading/survival | 76.6(76.2, 76.9) | 75.5(75.1, 75.9) |

Deep learning models were trained on AREDS and externally validated on AREDS2 as an independent cohort. In separate experiments, to externally validate the models on an independent dataset, we trained the models on AREDS (2,177 participants) and tested them on AREDS2 (1,121 participants). Table 5 shows that deep features/survival demonstrated the highest accuracy of five-year predictions in all scenarios, and DL grading/survival also had higher accuracy than retinal specialists/calculator.

TABLE 5

| | Tested on the |
|---|---|
| Models | entire AREDS2 |
| Late AMD | |
| Deep features/survival | 71.0(70.2, 71.7) |
| DL grading/survival | 69.7(68.9, 70.5) |
| Retinal specialists/calculator | 63.9(63.2, 64.6) |
| Retinal specialists/SSS* | 62.5(62.3, 62.7) |
| Geographic atrophy | |
| Deep features/survival | 75.3(74.5, 76.0) |
| DL grading/survival | 75.0(74.0, 76.0) |
| Retinal specialists/calculator | 64.4(63.6, 65.2) |
| Retinal specialists/SSS* | — |
| Neovascular AMD | |
| Deep features/survival | 62.8(61.9, 63.8) |
| DL grading/survival | 61.8(61.0, 62.7) |
| Retinal specialists/calculator | 61.8(60.8, 62.9) |
| Retinal specialists/SSS* | — |

*Retinal specialists/SSS-makes predictions at one fixed interval of five years and for late AMD only (i.e., not by disease subtype); unlike all other models, for SSS, late AMD is defined as NV or central GA (instead of NV or any GA)

Research software prototype was tested for AMD progression prediction. To demonstrate how these algorithms could be used in practice, we developed a software prototype that allows researchers to test our model with their own data. The application (shown in FIG. 6A) receives bilateral CFP and performs autonomous AMD classification and risk prediction. For transparency, the researcher is given (i) grading of drusen and pigmentary abnormalities, (ii) predicted SSS, and (iii) estimated risks of late AMD, GA, and NV, over 1-12 years. This approach allows improved transparency and flexibility: users may inspect the automated gradings, manually adjust these if necessary, and recalculate the progression risks. Following further validation, this software tool may potentially augment human research and clinical practice.

### Example 5: Statistical Analysis

As the primary outcome measure, the performance of the risk prediction models was assessed by the C-statistic at five years from study baseline. Five years from study baseline was chosen as the interval for the primary outcome measure since this is the only interval where comparison can be made with the SSS, and the longest interval where predictions can be tested using the AREDS2 data.

For binary outcomes such as progression to late AMD, the C-statistic represents the area under the receiver operating characteristic curve (AUC). The C-statistic is computed as follows: all possible pairs of participants are considered where one participant progressed to late AMD and the other participant in the pair progressed later or not at all; out of all these pairs, the C-statistic represents the proportion of pairs where the participant who had been assigned the higher risk score was the one who did progress or progressed earlier. A C-statistic of 0.5 indicates random predictions, while 1.0 indicates perfectly accurate predictions. We used 200 bootstrap samples to obtain a distribution of the C-statistic and reported 95% confidence intervals. For each bootstrap iteration, we sampled n patients with replacement from the test set of n patients.

As a secondary outcome measure of performance, the Brier score was calculated from prediction error curves. The Brier score is defined as the squared distances between the model's predicted probability and actual late AMD, GA, or NV status, where a score of 0.0 indicates a perfect match. The Wald test was used to assess the statistical significance of each factor in the survival models. It corresponds to the ratio of each regression coefficient to its standard error. The ‘survival’ package in R version 3.5.2 was used for Cox proportional hazards model evaluation. Finally, saliency maps were generated to represent the image locations that contributed most to decision-making by the DL models (for drusen or pigmentary abnormalities). This was done by back-projecting the last layer of the neural network. The Python package ‘keras-vis’ was used to generate the saliency map.

### Example 6: Evaluation of the Deep Learning RPD Models and Comparison with Human Clinical Practitioners

Each model was evaluated against the gold standard reading center grades of the test set. For each model, the following metrics were calculated: sensitivity (also known as recall), specificity, area under the curve (AUC) of the receiver operating characteristics (ROC), Cohen's kappa, accuracy, precision, and F1-score. Sensitivity, specificity, AUC, and kappa are evaluation metrics commonly used in clinical research, while AUC and F1-score (which incorporates sensitivity and precision into a single metric) are frequently used in image classification research. For each performance metric, the metric and its 95% confidence interval were calculated by bootstrap analysis (i.e., by randomly resampling instances with replacement 2,000 times to obtain a statistical distribution for each metric).

First, using the full test set, the performance of the five different CNNs was compared (separately for the FAF and the CFP task), with AUC as the primary performance metric and kappa as the secondary performance metric.

Second, the performance of the CNN with the highest AUC was compared (separately for the FAF and the CFP task) with the performance of four ophthalmologists with a special interest in RPD, who manually graded the images by viewing them on a computer screen at full image resolution. The ophthalmologists comprised two retinal specialists (in practice as a retinal specialist for 35 years (EC) and 2 years (TK)) and two post-residency retinal fellows (one second year (CH) and one first year (AT)). Prior to grading, all four ophthalmologists were provided with the same RPD imaging definitions as those used by the reading center graders (see above). In order to capture the usual daily practice of RPD grading by the ophthalmologists, no standardization exercise was performed prior to grading by the four ophthalmologists. In addition, for the CFP task only, comparison was also made with the CFP-based reading center grades. In all cases, the ground truth for the CFP images was the reading center label from the corresponding FAF images. For these comparisons, kappa was the primary performance metric and the other metrics (particularly accuracy and F1-score) were the secondary performance metrics. Kappa was selected because it handles data with unbalanced classes well (as in this study, where the number of negative instances outweighed the number of positive ones). These comparisons were conducted using a random sample (from the full test set) of 263 FAF images (from 50 participants) and the 263 corresponding CFP images; the four ophthalmologists each graded these images, independently of each other.

In secondary analyses, the performance of the CNN with the highest AUC was reanalyzed using the full test set but excluding eyes whose reading center grading changed over the study period from RPD absent to present. This secondary analysis was performed in order to explore scenarios in which performance might be higher or lower, specifically for the case of newly arising RPD. These RPD ‘in evolution’ are thought to be localized and subtle, such that some disagreement between the deep learning models and the reading center determination might be expected, regarding the exact time-points in successive images at which these early RPD are judged as definitely present.

Finally, attention maps were generated to investigate the image locations that contributed most to decision-making by the deep learning models. This was done by back-projecting the last layer of the neural network. The Python package ‘keras-vis’ was used to generate the attention maps.

### Example 7: Performance of Deep Learning Models in Detecting Reticular Pseudodrusen from Fundus Auto Fluorescence Images and Color Fundus Photographs: Comparison of Five Different Convolutional Neural Networks

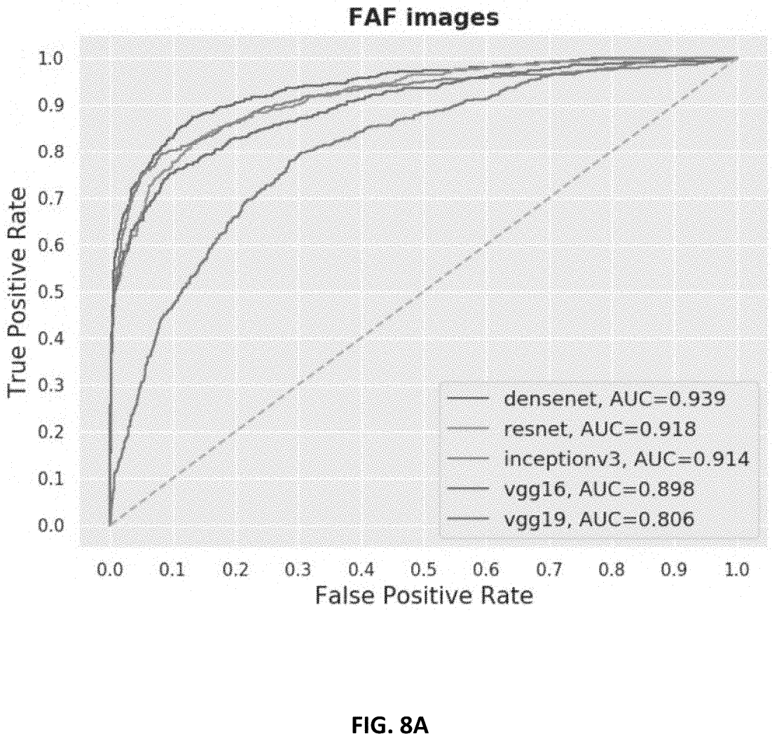

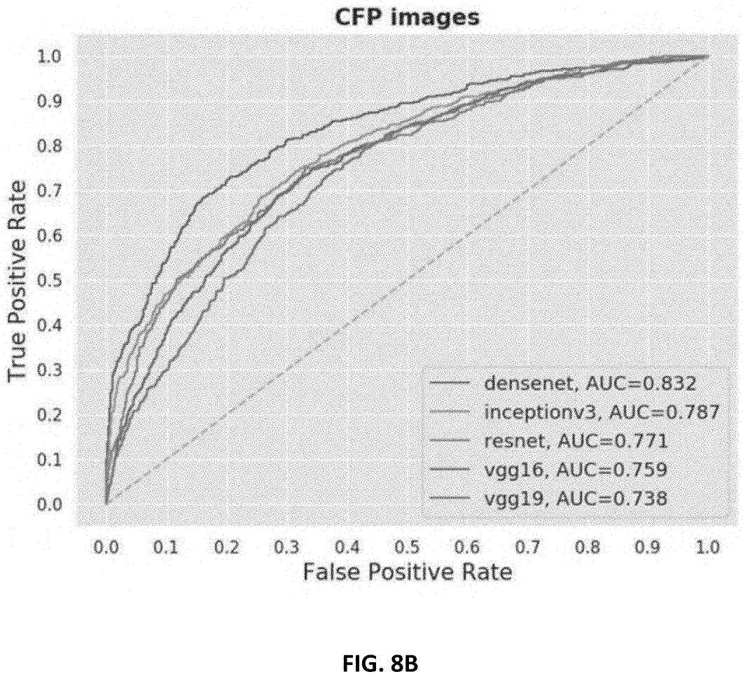

Each of five CNNs was used to analyze the FAF images in the test set. Their accuracy in detecting RPD presence, relative to the gold-standard (reading center grading of FAF images), was quantitated using multiple performance metrics. Separately, each of five CNNs was used to analyze CFP images in the test set. Their accuracy in detecting RPD presence, relative to the gold-standard (labels transferred from reading center grading of the corresponding FAF images), was quantitated in a similar way. The results are shown in Table 6 and in FIGS. 8A-8B.

TABLE 6
Performance metric (95% confidence interval)*

| | | | | | | |
|---|---|---|---|---|---|---|
| Area under | Sensitivity | | | | | |
| curve | (recall) | Specificity | Kappa | Accuracy | Precision | F1-score |

Fundus autofluorescence images

| | | | | | | | |
|---|---|---|---|---|---|---|---|
| VGG16 | 0.898 | 0.574 | 0.980 | 0.628 | 0.875 | 0.907 | 0.702 |
| | (0.881-0.913) | (0.534-0.612) | (0.972-0.986) | (0.589-0.666) | (0.861-0.889) | (0.876-0.935) | (0.669-0.733) |
| VGG19 | 0.806 | 0.142 | 0.983 | 0.169 | 0.766 | 0.738 | 0.238 |
| | (0.785-0.826) | (0.115-0.170) | (0.976-0.988) | (0.133-0.207) | (0.749-0.783) | (0.658-0.816) | (0.197-0.279) |
| lnceptionV3 | 0.914 | 0.686 | 0.968 | 0.706 | 0.896 | 0.882 | 0.772 |
| | (0.898-0.929) | (0.650-0.724) | (0.959-0.976) | (0.671-0.739) | (0.883-0.908) | (0.850-0.908) | (0.744-0.799) |
| ResNet | 0.918 | 0.618 | 0.961 | 0.635 | 0.873 | 0.846 | 0.714 |
| | (0.904-0.931) | (0.579-0.657) | (0.951-0.970) | (0.596-0.670) | (0.859-0.886) | (0.810-0.878) | (0.682-0.744) |
| DenseNet | 0.939 | 0.704 | 0.967 | 0.718 | 0.899 | 0.882 | 0.783 |
| | (0.927-0.950) | (0.667-0.741) | (0.958-0.975) | (0.685-0.751) | (0.887-0.911) | (0.851-0.909) | (0.755-0.809) |

Color fundus photographs

| | | | | | | | |
|---|---|---|---|---|---|---|---|
| VGG16 | 0.759 | 0.179 | 0.968 | 0.193 | 0.764 | 0.662 | 0.281 |
| | (0.736-0.782) | (0.150-0.212) | (0.960-0.976) | (0.154-0.235) | (0.748-0.782) | (0.588-0.736) | (0.240-0.326) |
| VGG19 | 0.738 | 0.256 | 0.932 | 0.229 | 0.757 | 0.566 | 0.353 |
| | (0.716-0.761) | (0.223-0.293) | (0.919-0.943) | (0.187-0.274) | (0.739-0.775) | (0.508-0.626) | (0.312-0.394) |
| lnceptionV3 | 0.787 | 0.475 | 0.891 | 0.393 | 0.783 | 0.603 | 0.531 |
| | (0.766-0.808) | (0.436-0.517) | (0.876-0.905) | (0.350-0.437) | (0.767-0.800) | (0.558-0.647) | (0.494-0.568) |
| ResNet | 0.779 | 0.252 | 0.963 | 0.273 | 0.779 | 0.707 | 0.372 |
| | (0.763-0.797) | (0.217-0.288) | (0.954-0.972) | (0.230-0.316) | (0.763-0.797) | (0.645-0.766) | (0.328-0.415) |
| DenseNet | 0.832 | 0.538 | 0.904 | 0.470 | 0.809 | 0.660 | 0.593 |
| | (0.812-0.851) | (0.498-0.575) | (0.889-0.918) | (0.426-0.511) | (0.793-0.825) | (0.618-0.705) | (0.557-0.627) |

*The performance metrics and their 95% confidence intervals were evaluated by bootstrap analysis.

For the FAF image analyses, DenseNet, relative to the other four CNNs, achieved the highest AUC, the primary performance metric, at 0.939 (95% confidence interval 0.927-0.950), and the highest kappa, the secondary performance metric, at 0.718 (0.685-0.751). Of the five other performance metrics, it was highest for three (sensitivity, accuracy, and F1-score), relative to the other CNNs. It achieved sensitivity 0.704 (0.667-0.741) and specificity 0.967 (0.958-0.975).

For the CFP image analyses, DenseNet achieved the highest AUC, at 0.832 (0.812-0.851), and the highest kappa, at 0.470 (0.426-0.511). Of the five other performance metrics, it was highest for four (sensitivity, accuracy, precision, and F1-score), relative to the other CNNs. It achieved sensitivity 0.538 (0.498-0.575) and specificity 0.904 (0.889-0.918).

### Example 8: Performance of Automated Deep Learning Models Versus Human Practitioners in Detecting Reticular Pseudodrusen

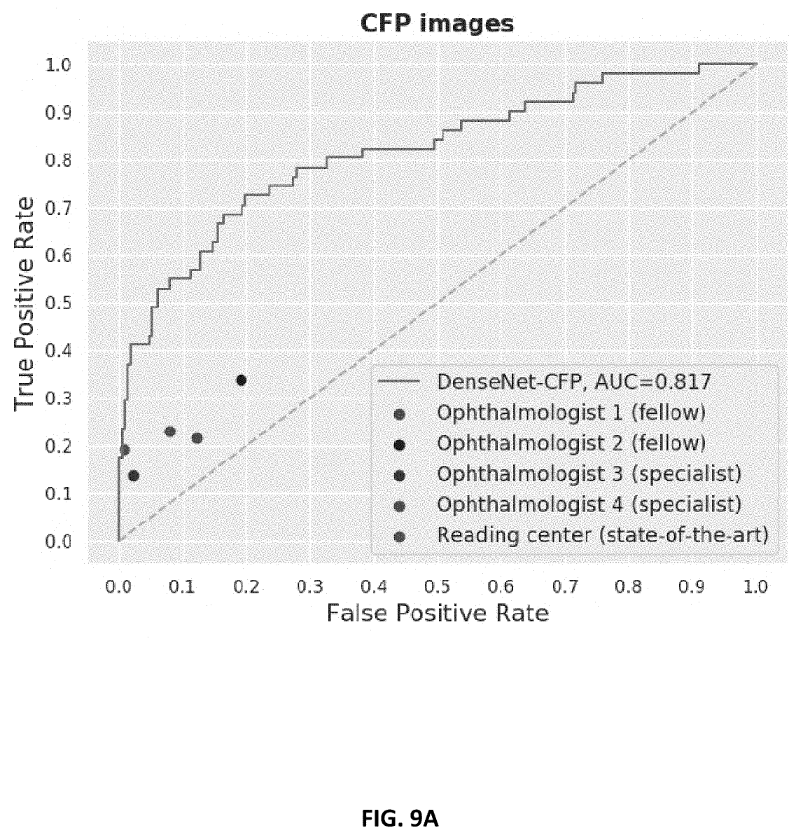

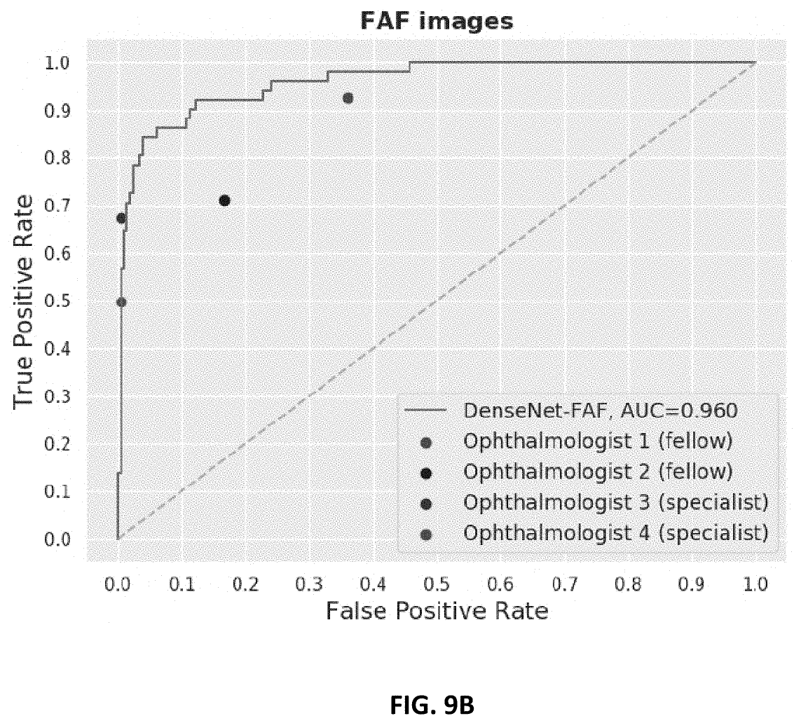

The highest performing CNN, DenseNet, was used to analyze images from a random subset of the test set (263 FAF and CFP corresponding image pairs). For both FAF and CFP images in this test set, the performance metrics for the detection of RPD presence were compared to those obtained by each of four ophthalmologists who manually graded the images (when viewed on a computer screen at full image resolution). For the CFP images only, the performance metrics were also compared with CFP-derived labels (i.e., reading center grading of the CFP images), using the FAF-derived labels as the ground truth. The results are shown in Table 7. In addition, the ROC curves for the two DenseNet deep learning models are shown in FIGS. 9A-9B. For comparison, the performance of the four ophthalmologists is shown as four single points; for the CFP task, the performance of the reading center grading is also shown as a single point.

TABLE 7
Performance metric (95% confidence interval)*

| | | | | | | |
|---|---|---|---|---|---|---|
| Area | | | | | | |
| under | Sensitivity | | | | | |
| curve | (recall) | Specificity | Kappa | Accuracy | Precision | F1 -score |

Fundus autofluorescence images

| | | | | | | | |
|---|---|---|---|---|---|---|---|
| Ophthalmologist 1 | — | 0.929 | 0.641 | 0.367 | 0.696 | 0.380 | 0.539 |
| (2^{nd} year fellow) | | (0.854-0.984) | (0.577-0.704) | (0.277-0.462) | (0.641-0.752) | (0.303-0.471) | (0.452-0.629) |
| Ophthalmologist 2 | — | 0.712 | 0.834 | 0.472 | 0.811 | 0.508 | 0.591 |
| (1^{st} year fellow) | | (0.583-0.833) | (0.781-0.883) | (0.346-0.602) | (0.759-0.856) | (0.389-0.640) | (0.482-0.698) |
| Ophthalmologist 3 | — | 0.498 | 0.995 | 0.601 | 0.899 | 0.961 | 0.653 |
| (retinal specialist for | | (0.367-0632) | (0.986-1.000) | (0.464-0.723) | (0.863-0.933) | (0.870-1.000) | (0.532-0.766) |
| 35 y) | | | | | | | |
| Ophthalmologist 4 | — | 0.673 | 0.995 | 0.756 | 0.933 | 0.973 | 0.795 |
| (retinal specialist for 2 y) | | (0.544-0.800) | (0.986-1.000) | (0.641-0.855) | (0.900-0.961) | (0.906-1.000) | (0.693-0.883) |
| DenseNet | 0.962 | 0.776 | 0.977 | 0.789 | 0.937 | 0.894 | 0.828 |
| | | (0.646-0.874) | (0.955-0.995) | (0.675-0.875) | (0.907-0.963) | (0.787-0.977) | (0.725-0.898) |

Color fundus photographs

| | | | | | | | |
|---|---|---|---|---|---|---|---|
| Ophthalmologist 1 | — | 0.217 | 0.878 | 0.105 | 0.750 | 0.299 | 0.249 |
| (2^{nd} year fellow) | | (0.109-0.345) | (0.832-0.920) | (0.000-0.802) | (0.700-0.802) | (0.151-0.447) | (0.129-0.358) |
| Ophthalmologist 2 | — | 0.337 | 0.808 | 0.138 | 0.717 | 0.297 | 0.314 |
| (1^{st} year fellow) | | (0.214-0.459) | (0.751-0.856) | (0.019-0.267) | (0.662-0.768) | (0.194-0.414) | (0.210-0.425) |
| Ophthalmologist 3 | | 0.137 | 0.976 | 0.159 | 0.814 | 0 589 | 0.217 |
| (retinal specialist for | | (0.053-0.226) | (0.952-0.995) | (0.040-0.279) | (0.768-0.855) | (0.300-0.882) | (0.089-0.341) |
| 35 y) | | | | | | | |
| Ophthalmologist 4 | — | 0.232 | 0.919 | 0.180 | 0.787 | 0.410 | 0.293 |
| (retinal specialist for 2 y) | | (0.125-0.345) | (0.882-0.954) | (0.052-0.308) | (0.741-0.833) | (0.227-0.575) | (0.168-0.410) |
| Reading centert | — | 0.193 | 0.991 | 0.258 | 0.836 | 0.837 | 0.311 |
| | | (0.096-0.296) | (0.976-1.000) | (0.130-0.387) | (0.791-0.875) | (0.581-1.000) | (0.173-0.444) |
| DenseNet | 0.817 | 0.527 | 0.920 | 0.471 | 0.844 | 0.614 | 0.565 |
| | | (0.398-0.670) | (0.885-0.954) | (0.330-0.606) | (0.798-0.886) | (0.473-0.756) | (0.434-0.674) |

*The performance metrics and their 95% confidence intervals were evaluated by bootstrap analysis.
†Reading center evaluation of color fundus photographs, assessed by the ground truth of reading center independent evaluation of the corresponding fundus autofluorescence images.

For the FAF image analyses, DenseNet, relative to the four ophthalmologists, achieved the highest kappa, the primary performance metric, at 0.789 (0.675-0.875). This was numerically higher than the kappa of one retinal specialist, substantially higher than that of the other retinal specialist, and very substantially higher than that of the two retinal fellows. Regarding accuracy and F1-score, DenseNet achieved the highest performance, at 0.937 (0.907-0.963) and 0.828 (0.725-0.898), respectively. The two retinal specialists demonstrated high levels of specificity (0.995 (0.986-1.000) for both) and precision (0.961 (0.870-1.000) and 0.973 (0.906-1.000)), but at the expense of decreased sensitivity (0.498 (0.367-0.632) and 0.673 (0.544-0.800)). Regarding the ROC curves (FIG. 9A), the performance of the two retinal specialists was similar or very slightly superior to that of DenseNet (AUC 0.962), while the performance of the two fellows was either moderately or substantially inferior.

For the CFP image analyses, DenseNet, relative to the four ophthalmologists, achieved the highest kappa, at 0.471 (0.330-0.606), the primary performance metric. This was very substantially higher than the kappa of all four ophthalmologists (range 0.105-0.180). It was also substantially higher than the reading center grading of CFP, at 0.258 (0.130-0.387). Regarding accuracy and F1-score, DenseNet achieved the highest performance, at 0.844 (0.798-0.886) and 0.565 (0.434-0.674), respectively. Both the four ophthalmologists and the reading center grading demonstrated relatively high levels of specificity but extremely low levels of sensitivity, consistent with the idea that RPD are not consistently discernable from CFP by human viewers. Regarding the ROC curves (FIG. 9B), the performance of DenseNet (AUC 0.817) was substantially superior to that of all four ophthalmologists, and very substantially superior to that of three of them.

### Example 9: Secondary Analyses

In order to explore scenarios in which the DenseNet deep learning models might perform more or less accurately, pre-specified secondary analyses were performed, considering eyes with RPD ‘in evolution’ (i.e., where the reading center grading for RPD on FAF images changed from absent to present over the course of the AREDS2 follow-up period). From previous natural history studies, newly arisen RPD are thought to be localized (rather than widespread across the macula), and may be subtle, even on FAF imaging. Hence, the test set was divided (at the eye level) into two subgroups, for both the FAF images and the CFP: (i) images from eyes whose RPD grading changed from absent to present during follow-up, and (ii) all other images. The performance of the two DenseNet deep learning models was tested separately on the two subgroups of images. The performance metrics are shown in Table 8.

TABLE 8
Performance metric

| | | | | | | |
|---|---|---|---|---|---|---|
| Area | | | | | | |
| under | Sensitivity | | | | | |
| curve | (recall) | Specificity | Kappa | Accuracy | Precision | F1-score |

Fundus autofluorescence images

| | | | | | | | |
|---|---|---|---|---|---|---|---|
| All images (n = 2,249) | 0.941 | 0.707 | 0.967 | 0.721 | 0.900 | 0.882 | 0.785 |
| Images from converting eyes | 0.827 | 0.482 | 0.929 | 0.383 | 0.675 | 0.900 | 0.628 |
| only (n = 197) | | | | | | | |
| All images except those from | 0.960 | 0.761 | 0.969 | 0.766 | 0.922 | 0.879 | 0.816 |
| converting eyes (n = 2,052) | | | | | | | |

Color fundus photographs

| | | | | | | | |
|---|---|---|---|---|---|---|---|
| All images (n = 2,249) | 0.832 | 0.537 | 0.903 | 0.469 | 0.809 | 0.660 | 0.592 |
| Images from converting eyes | 0.636 | 0.348 | 0.847 | 0.180 | 0.563 | 0.750 | 0.476 |
| only (n = 197) | | | | | | | |
| All images except those from | 0.863 | 0.582 | 0.907 | 0.507 | 0.832 | 0.649 | 0.613 |
| converting eyes (n = 2,052) | | | | | | | |

For both the FAF and the CFP analyses, as hypothesized, the performance metrics were generally inferior for subgroup (i) (i.e., eyes with RPD in evolution) and superior for subgroup (ii). For example, for the FAF image analyses, the AUC was 0.827 versus 0.960, respectively, and the kappa was 0.383 versus 0.766, respectively. Similarly, for the CFP analyses, the AUC was 0.636 and 0.863, respectively, and the kappa was 0.180 and 0.507, respectively.

In addition, post hoc analyses were performed to explore whether the performance of the two DenseNet models was affected by AMD severity, specifically the presence or absence of late AMD.

### Example 10: Attention Maps Generated on Fundus Autofluorescence Images and Color Fundus Photographs by Deep Learning Model Evaluation

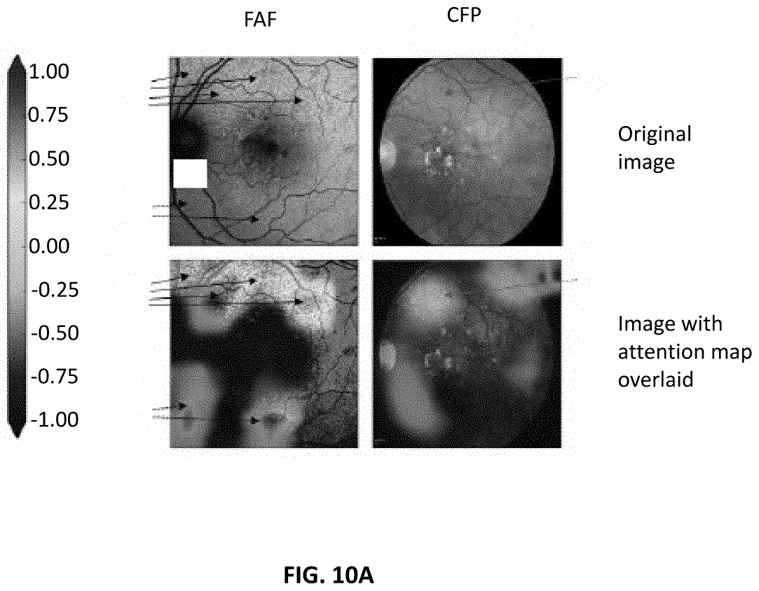

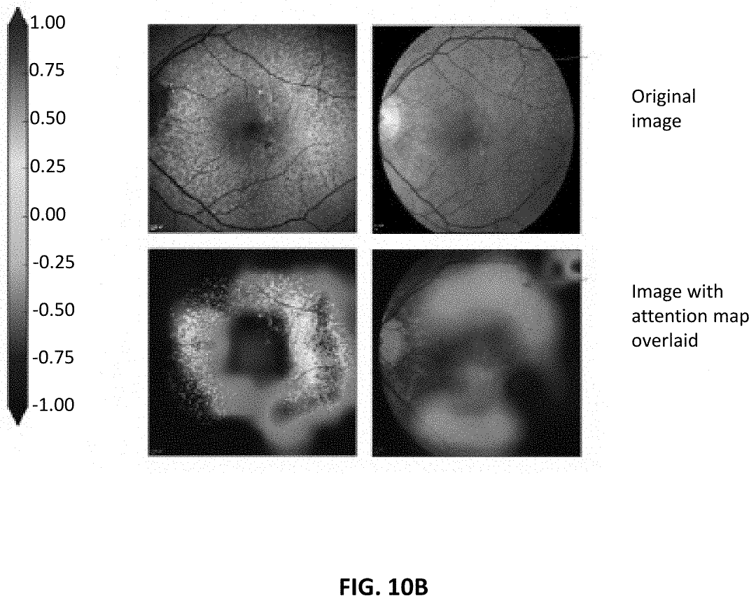

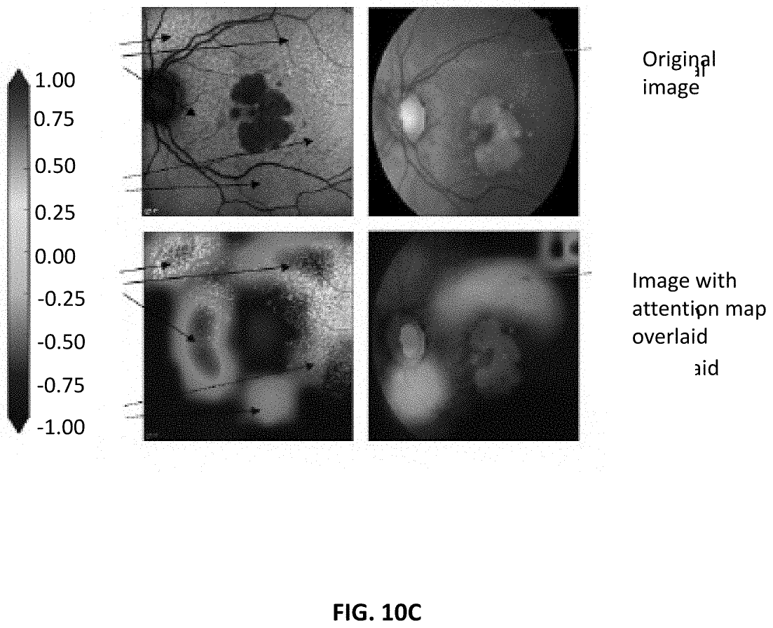

Consequent to the evaluation of FAF and CFP images using the DenseNet models, attention maps were generated and superimposed on the fundus images, to represent in a quantitative manner the relative contributions that different areas in each image made to the ascertainment decision. Examples of these attention maps are shown for three separate FAF-CFP image pairs from different study participants in FIGS. 10A-10C.

FIGS. 10A-10C show the study participants' original FAF image in the top left, the FAF image with the deep learning attention map overlaid in the bottom left, the original CFP image in the top right, and the CFP image with the deep learning attention map overlaid.

In FIG. 10A, reticular pseudodrusen (RPD) are observed in the original FAF image as ribbon-like patterns of round and oval hypoautofluorescent lesions with intervening areas of normal and increased autofluorescence in the following locations: (i) in an arcuate band across the superior macula (black arrows), extending across the vascular arcade to the area superior to the optic disc, and, less prominently and affecting a smaller area, (ii) in the inferior macula (broken black arrows), extending across the vascular arcade to the area inferior to the optic disc. For the FAF attention map, the areas of highest signal correspond very well with the retinal locations observed to contain RPD in the original FAF image. The superior arcuate band, extending to the area superior to the optic disc, is demonstrated clearly (black arrows), as are the two inferior locations (broken black arrows). The predominance of superior over inferior macular involvement is also captured in the attention map. For the CFP image, RPD are not observed in the majority of retinal locations known from the corresponding FAF image known to be affected. However, an area of possible involvement may be present on the CFP at the superonasal macula (broken gray arrow). For the CFP attention map, the areas of highest signal correspond very well with both (i) the retinal locations observed to contain RPD in the original FAF image (even though the deep learning model never received the FAF image as input), and (ii) the areas of highest signal in the FAF image attention map. Hence, the CFP attention map has areas of high signal in both (i) the area (superonasal macula) with RPD possibly visible to humans on the CFP (broken gray arrow), and (ii) other areas where RPD appear invisible on the CFP but visible on the corresponding FAF image.

In FIG. 10B, RPD are observed in the original FAF image clearly as widespread ribbon-like patterns of round and oval hypoautofluorescent lesions with intervening areas of normal and increased autofluorescence. The area affected is large, affecting almost the whole macula but sparing the central macula, i.e., in a doughnut configuration. For the FAF attention map, the doughnut configuration is captured well on the attention map, i.e., the areas of highest signal correspond very well with the retinal locations observed to contain RPD in the FAF image. In the original CFP image, RPD are observed in some but not all of the retinal locations known from the corresponding FAF image to contain RPD: the superior peripheral macula appears to contain RPD (broken gray arrow), but they are not clearly visible in the inferior peripheral macula. For the CFP with deep learning attention map, the CFP attention map has areas of high signal in both (i) the area (superior macula) with RPD visible to humans on the CFP (broken gray arrow), and (ii) other areas (inferior macula) where RPD appear invisible on the CFP but visible on the corresponding FAF image (even though the deep learning model never received the FAF image as input).

In FIG. 10C, RPD are observed in the original FAF image as ribbon-like patterns of round and oval hypoautofluorescent lesions with intervening areas of normal and increased autofluorescence. These are relatively widespread across the macula (black arrows; corresponding almost to a doughnut configuration), as well as the area superior to the optic disc (black arrow), but are less apparent in the inferior and inferotemporal macula (broken black arrows). In the FAF image with the overlaid attention map, the areas of highest signal correspond very well with the retinal locations observed to contain RPD in the original FAF image (black arrows). The doughnut configuration, with partial sparing of the inferotemporal macula (broken black arrows), complete sparing of the central macula (affected by geographic atrophy), and additional involvement of the area superior to the optic disc, is captured well. RPD are not observed in the original CFP image in the majority of retinal locations known from the corresponding FAF image known to be affected. However, an area of possible involvement may be present on the CFP at the superotemporal macula (broken gray arrow). The CFP attention map has areas of high signal in both (i) the area (superotemporal macula) with RPD potentially visible to humans on the CFP (broken gray arrow), and (ii) another area (inferior to the optic disc) that might potentially contain additional signatures of RPD presence.

As seen in these examples, the areas of the fundus images that contributed most consequentially to RPD detection were located in the outer areas of the central macula within the vascular arcades, approximately 3-4 mm from the foveal center. Despite the fact that the algorithms were not subject to any spatial guidance with respect to the location of RPD, these ‘high attention’ areas correspond well to the typical localization of clinically observable RPD within fundus images. In the specific examples shown in FIGS. 10A-10C, clinically observable RPD can be located within these high attention areas. Hence, through these attention maps, the outputs of the deep learning models display a degree of face validity and interpretability for the detection of RPD. Qualitatively, there was a moderate degree of correspondence between the attention maps of corresponding FAF-CFP pairs, with each showing a similar macular distribution.

### Example 11: Exploratory Error Analyses

Exploratory error analyses were performed in order to investigate the possibility that poor image quality may be a factor that limits deep learning model performance, and to observe potential patterns of findings in images that were associated with errors. These analyses were performed by examining random samples of 20 images in each of the four following categories of images misclassified by the two DenseNet deep learning models: FAF image false negatives; false positives; CFP image false negatives; false positives; representative examples for each of the error categories are shown in the Supplementary Material. The images were analyzed qualitatively. Regarding the FAF images, 10 of the false negative and two of the false positive cases had poor/very poor image quality. For three of the false positive cases, the reading center label was negative for those particular images, but positive for subsequent images acquired 1-2 years later. Regarding the CFP, seven of the false positive and five of the false negative cases had poor/very poor image quality. These error analyses suggest that overall model accuracy may be negatively impacted by a subset of images of poor quality. While we did not filter images by quality criteria in the current study, the institution of such measurements may be used to increase model performance.

### Example 12: External Validation of the Method to Detect RPD

The method of detecting RPD from retinal images was externally validated by measuring its performance on images from a completely different population on a different continent: participants in the population-based Rotterdam Eye Study (Rotterdam, Netherlands). In brief, the study population consisted of 278 eyes from 230 participants. Of the 278 eyes, 73 were positive for RPD and the remaining 205 were negative (including 113 eyes with soft drusen but no RPD and 92 control eyes with neither soft drusen nor RPD). The ground truth used to measure performance were the labels (RPD present/absent) from reading center grading (EyeNED Reading Center, Rotterdam), based on the combination of CFP, FAF, and near-infrared imaging.

On this external test set selected to have a high prevalence of RPD in the context of AMD, the performance metrics of the deep learning method for detecting RPD were: Area under the curve (AUC)=0.986; Accuracy=0.942; Sensitivity=0.849; Specificity=0.976; Precision=0.925; F-score=0.886.

These performance metrics were superior to those observed during internal validation on the AREDS2 test set. Hence, external validation was highly successful. This shows that the method of detecting RPD is generalizable to other populations beyond the specific population on which it was trained.

### Example 13: Multi-Modal, Multi-Task, Multi-Attention (M3) Deep Learning Framework

Three non-M3 deep learning models were created, one for each image scenario, in order to compare performance between these and the M3 models. Importantly, the structure of the non-M3 CFP model and the non-M3 FAF model represents what is used in existing studies of other medical computer vision tasks to achieve state-of-the-art performance. Hence, the non-M3 models created were expected to have a high level of performance, in order to set a high standard for the M3 models. For the CFP-only and FAF-only image scenarios, the non-M3 model comprised a CNN backbone, followed by the fully-connected and output layers. To ensure a fair comparison, InceptionV3 was used as the CNN backbone for both the non-M3 and the M3 models. InceptionV3 is a state-of-the-art CNN architecture that is used commonly in medical computer vision applications. For the same reasons, the fully-connected and output layers were exactly the same as those used in the M3 models. For the CFP-FAF image scenario, the non-M3 model used a typical concatenation to combine the CFP and FAF image features from the InceptionV3 CNN backbones. Unlike the M3 models, the three non-M3 models were trained separately and did not use attention mechanisms.

For each of the three image scenarios, an M3 model was trained 10 times using the same training/validation/test split shown in Table 1, to create 10 individual M3 models (i.e. 30 models in total). Similarly, each non-M3 model was trained 10 times (i.e. another 30 models), using the same training/validation/test split. This was to allow a fair comparison between the two model types, including meaningful statistical analysis (as described below). Both the M3 and the non-M3 models shared the same hyperparameters and training procedures to ensure a fair comparison (except that the M3 models had an additional cascading task fine-tuning step, as shown in FIG. 11). The InceptionV3 CNN backbones were pre-trained using ImageNet, an image database of over 14 million natural images with corresponding labels, using methods described previously. During the training process, each input image was scaled to 512×512 pixels. The model parameters were updated using the Adam optimizer (learning rate of 0.001) for every minibatch of 16 images. An early stop procedure was applied to avoid overfitting: the training was stopped if the loss on the validation set no longer decreased for 5 epochs. The M3 models completed training within 30 epochs, whereas the non-M3 model completed training within 10 epochs. In addition, image augmentation procedures were used, as follows, in order to increase the dataset size and to strengthen model generalizability: (i) rotation (0-180 degree), (ii) horizontal flip, and (iii) vertical flip. For the cascading task fine-tuning step of the M3 models, the same hyperparameters were used except for a learning rate of 0.0001. The models were implemented using Keras and TensorFlow. All experiments were conducted on a server with 32 Intel Xeon CPUs, using three NVIDIA GeForce GTX 1080 Ti 11 Gb GPUs for training and testing, with 512 Gb available in RAM memory.

### Example 14: Evaluation of the Deep Learning Models in Comparison with Each Other

For the RPD feature, each model was evaluated against the gold standard reading center grades on the full test set of images. For each model, the following metrics were calculated: F1-score, area under receiver operating characteristic (AUROC), sensitivity (also known as recall), specificity, Cohen's kappa, accuracy, and precision. The F1-score (which incorporates sensitivity and precision into a single metric) was the primary performance metric. The AUROC was the secondary performance metric. The performance of the deep learning models was evaluated separately for the three imaging scenarios; for each scenario, the performance of the M3 models was compared with those of the non-M3 models. The Wilcoxon rank sum test was used to compare the F1-scores of the 10 M3 and 10 non-M3 models (separately for each imaging modality). In addition, the differential performance of the models was analyzed by examining the distribution of cases correctly classified by both models, neither model, the non-M3 model only, or the M3 model only. For these analyses, bootstrapping was performed with 50 iterations, with one of the 10 models selected randomly for each iteration. Similar methods were followed for the other two AMD features (geographic atrophy and pigmentary abnormalities).

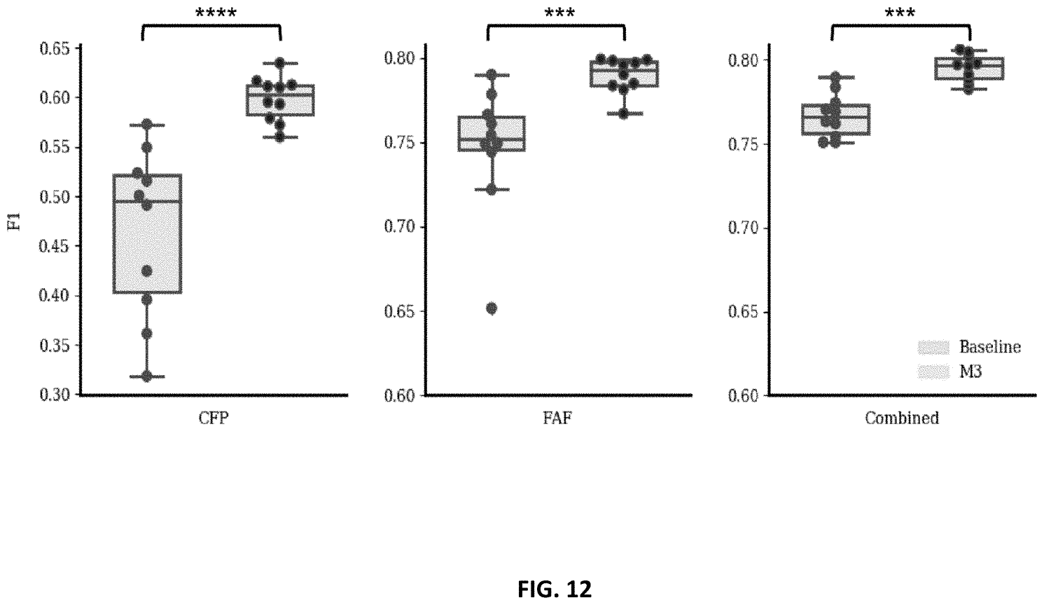

FIG. 12 shows box plots showing the F1 score results of the multi-modal, multi-task, multi-attention (M3) and standard (non-M3) deep learning convolutional neural networks for the detection of reticular pseudodrusen from color fundus photographs (CFP) alone, their corresponding fundus autofluorescence (FAF) images alone, or the CFP-FAF image pairs, using the full test set. Each model was trained and tested 10 times (i.e. 60 models in total), using the same training and testing images each time. The F1-scores were substantially higher for the FAF and CFP-FAF scenarios than for the CFP scenario. In all three image scenarios, the F1-score of the M3 model was significantly and substantially higher than that of the non-M3 model. This was particularly noticeable for the clinically important CFP scenario, with an increase of over 20% in the F1-score for the M3 model versus the non-M3 model (60.28 vs. 49.60; p<0.0001). In the FAF scenario, the median F1-scores were 79.30 (IQR 1.41) and 75.18 (IQR 1.94), respectively (p<0.001). In the CFP-FAF scenario, the median F1-scores were 79.67 (IQR 1.19) and 76.62 (IQR 1.71), respectively (p<0.001). The F1-score of the most accurate M3 model, among all runs, was 63.45 for CFP, 79.91 for FAF, and 80.61 for CFP-FAF. The equivalent AUROC values were 84.20, 93.55, and 93.76, respectively. Model calibration analyses were also performed.

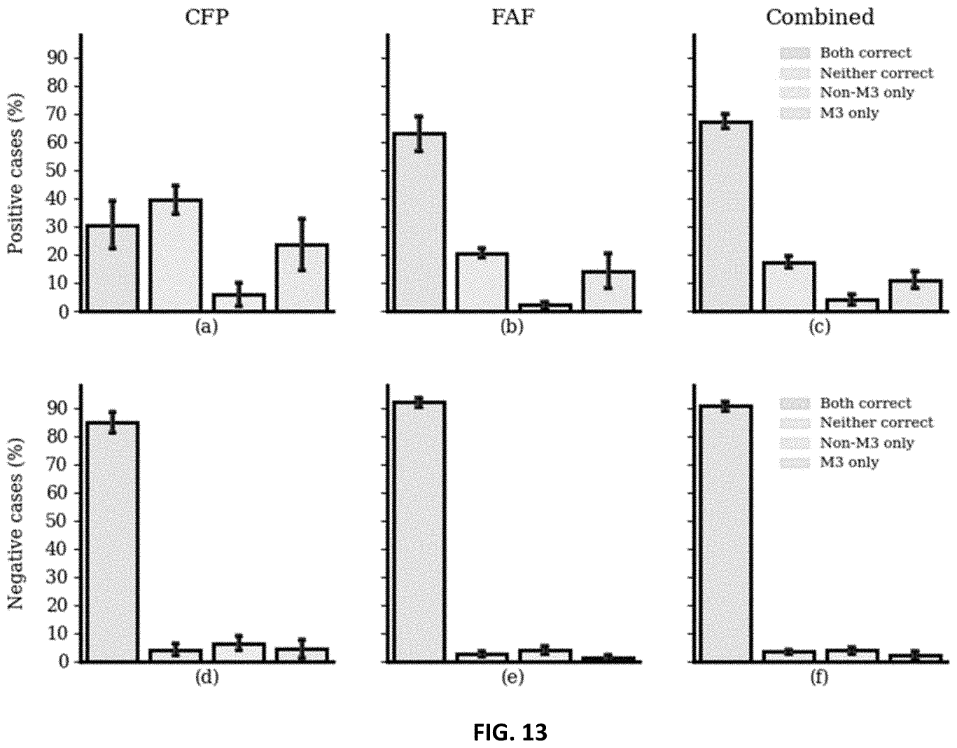

Table 9 shows performance results of the M3 and non-M3 deep learning convolutional neural networks for the detection of reticular pseudodrusen from color fundus photographs (CFP) alone, their corresponding fundus autofluorescence (FAF) images alone, or the CFP-FAF image pairs, using the full test set. The median and interquartile range (brackets) are shown for each performance metric. As observed in Table 9, using the same default cut-off threshold of 0.5, the sensitivity of the M3 models was substantially higher for all three image scenarios, and particularly for the CFP scenario. In addition, the M3 had higher AUROC for all three image scenarios, suggesting that the M3 could better distinguish positive and negative cases. The differential performance of the models was further analyzed by examining the distribution of cases correctly classified by both models, neither model, the M3 model only, or the non-M3 model only, as shown in FIG. 13. Analysis of the positive cases demonstrated a relatively high frequency where only the M3 model was correct, particularly for the CFP image scenario (mean 23.7%, SD 9.1%), and a very low frequency of cases where only the non-M3 model was correct (mean 6.1%, SD 4.1%). Similarly, in the FAF scenario, the equivalent figures were 14.2% (SD 6.1%) and 2.1% (SD 1.1%), respectively.

TABLE 9

| | | | | | | |
|---|---|---|---|---|---|---|
| | | Sensitivity | | | | |
| F1-score | Precision | (Recall) | Specificity | AUROC | Kappa | Accuracy |

CFP modality

| | | | | | | | |
|---|---|---|---|---|---|---|---|
| Standard (non-M3) | 49.60 (11.87) | 61.72 (13.00) | 42.13 (18.21) | 90.40 (7.55) | 77.50 (3.10) | 33.87 (10.00) | 76.74 (1.72) |
| M3 | 60.28 (2.98) | 66.39 (3.11) | 55.28 (9.21) | 89.62 (2.54) | 82 17 (1.05) | 46.49 (2.22) | 79.71 (1.02) |

FAF modality

| | | | | | | | |
|---|---|---|---|---|---|---|---|
| Standard (non-M3) | 75.18 (1.94) | 86.32 (4.15) | 67.13 (6.84) | 95.94 (1.72) | 91.39 (1.08) | 67.31 (2.63) | 87.76 (1.37) |
| M3 | 79.30 (1.41) | 81.90 (2.91) | 76.72 (3.99) | 93.64 (1.62) | 93.06 (0.49) | 71.71 (2.27) | 88.83 (1.02) |

Combined modality

| | | | | | | | |
|---|---|---|---|---|---|---|---|
| Standard (non-M3) | 76.62 (1.71) | 81.93 (6.86) | 71.66 (5.39) | 93.85 (2.69) | 91.53 (0.41) | 68.16 (2.11) | 87.97 (0.92) |
| M3 | 79.67 (1.19) | 80.38 (4.18) | 79.42 (3.39) | 92.70 (2.40) | 93.30 (0.46) | 71.90 (1.78) | 88.80 (0.80) |

In order to assess whether the multi-modal/multi-task or the multi-attention mechanism contributed most to improved performance of the M3 models, the performance of non-M3 models with one or the other mechanism was examined. The F1-score had an absolute increase of 10% (multi-modal/multi-task only) and 6% (multi-attention only), which suggests that both aspects contributed to improved performance (while multi-attention operation also improves model interpretability).

### Example 15: Evaluation of the Deep Learning Models in Comparison with Human Ophthalmologists

For the RPD feature, for each of the three image scenarios, the performance of the deep learning models was compared with the performance of 13 ophthalmologists who manually graded the same images (when viewed on a computer screen at full image resolution). For this comparison, the test set of images was a random subset of the full test set (at the participant level) and comprised 100 CFP, and the 100 corresponding FAF images, from 100 different participants (comprising 68 positive cases and 32 negative cases). Each model was trained and tested 10 times, using the same training and testing images each time. The ophthalmologists performed the grading independently of each other, and separately for the two image scenarios (i.e. CFP-alone then FAF-alone). The ophthalmologists comprised three different levels of seniority and specialization in retinal disease: ‘attending’ level (highest seniority) specializing in retinal disease (4 people), attending level not specializing in retinal disease (4 people), and ‘fellow’ level (lowest seniority) (5 people). Prior to grading, all the ophthalmologists were provided with the same RPD imaging definitions as those used by the reading center graders (i.e. as described above). The performance metrics were calculated, the Wilcoxon rank sum test applied, and ROC curves generated, as above.

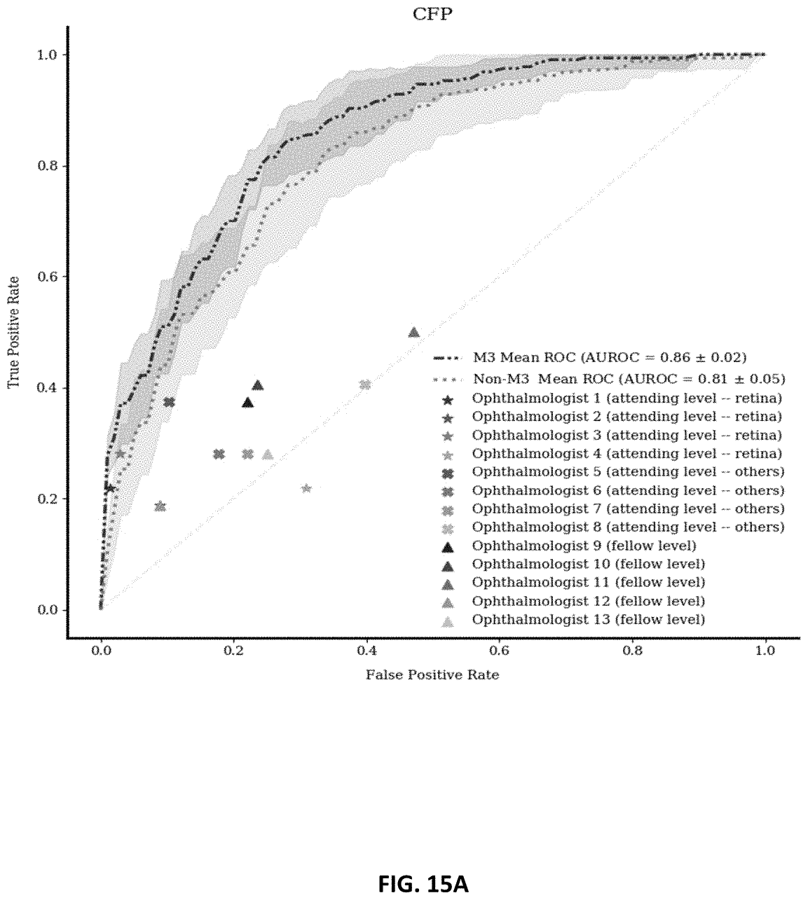

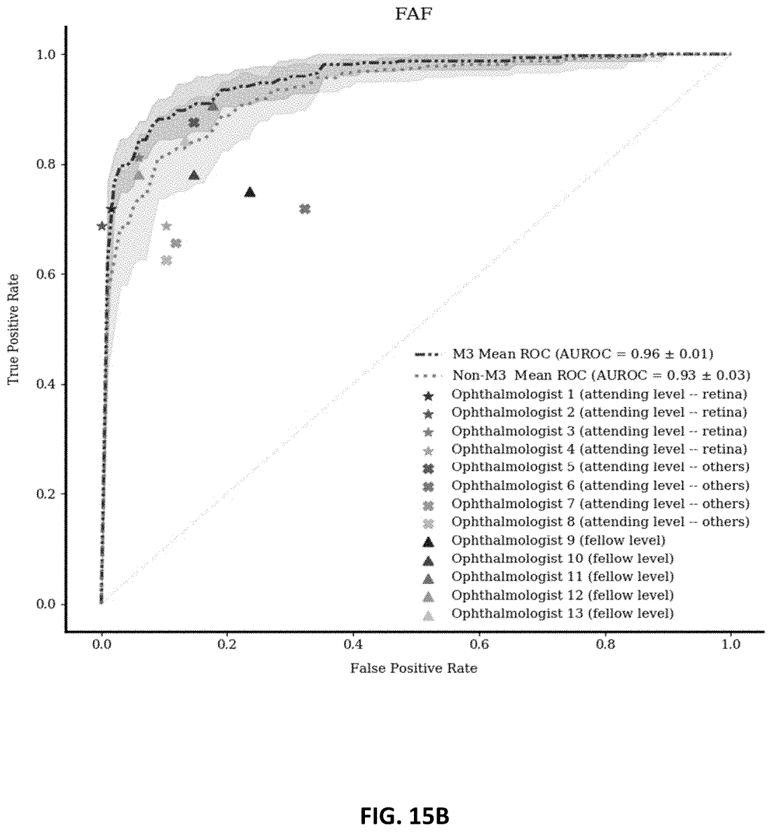

The results are shown in FIGS. 15A and 15B and Table 10. In FIGS. 15A and 15B, the performance of the deep learning models is shown by their ROC curves, with the performance of each ophthalmologist shown as a single point. For the CFP scenario (FIG. 15A), the median F1-scores of the ophthalmologists were 31.14 (IQR 10.43), 35.04 (IQR 5.34), and 40.00 (IQR 9.64), for the attending (retina), attending (non-retina), and fellow levels, respectively. This low level of human performance was expected, since RPD are typically observed very poorly on CFP, even at the gold standard level of reading center experts. In comparison, the median F1-score was 64.35 (IQR 6.29) for the M3 models and 49.14 (IQR 24.58) for the non-M3 models. Considering all 13 ophthalmologists together, the F1-scores of the M3 models were approximately 84% higher than those of the ophthalmologists (p<0.0001). Indeed, the performance of the M3 models was twice as high as that of the retinal specialists at attending level (the most senior level of ophthalmologists and those specialized in retinal disease).

For the FAF-alone image scenario (FIG. 15B), the median F1-scores of the ophthalmologists were 81.81 (IQR 3.43), 68.32 (IQR 5.86), and 79.41 (IQR 4.83), for the attending (retina), attending (non-retina), and fellow levels, respectively. In comparison, the median F1-score was 85.25 (IQR 5.24) for the M3 models and 78.51 (IQR 8.51) for the non-M3 models. Considering all 13 ophthalmologists together, the F1-scores of the M3 models were significantly higher than those of the ophthalmologists (p<0.001). By contrast, this was not true of the non-M3 models (p=0.95). Similarly, the performance of the M3 models was substantially superior to that of all three levels of ophthalmologists considered separately, including the most senior and specialized in retinal disease. Again, this was not true of the non-M3 models.

Table 10 provides performance results of the M3 and non-M) deep learning convolutional neural networks, in comparison with those of 13 ophthalmologists, for the detection of reticular pseudodrusen from color fundus photographs (CFP) alone or their corresponding fundus autofluorescence (FAF) images alone, using a random subset of the test set. The median and interquartile range (brackets) are shown for each performance metric.

TABLE 10

| | | | Sensitivity | | | | |
|---|---|---|---|---|---|---|---|
| | F1-score | Precision | (Recall) | Specificity | AUROC | Kappa | Accuracy |
| CFP modality | | | | | | | |
| Human level | | | | | | | |
| Fellow | 40.00 (9.64) | 44.44 (10.21) | 37.50 (12.50) | 76.47 (2.94) | — | 11.89 (12.82) | 65.00 (5.00) |
| Attending Other | 35.04 (5.34) | 40.18 (11.68) | 32.81 (10.16) | 80.15 (10.66) | — | 9.01 (11.18) | 63.50 (7.00) |
| Attending Retina | 31.14 (10.43) | 65.91 (39.49) | 21.88 (2.34) | 94.12 (11.76) | — | 18.68 (20.12) | 71.00 (9.75) |
| Overall | 35.00 (9.64) | 44.44 (15.38) | 28.12 (15.62) | 77.94 (16.18) | — | 11.89 (14.24) | 65.00 (8.00) |
| Model level | | | | | | | |
| Standard (non-M3) | 49.14 (24.58) | 71.43 (17.13) | 43.75 (27.34) | 91.18 (10.66) | 82.58 (5.09) | 29.28 (20.40) | 73.00 (6.25) |
| M3 | 64.35 (6.29) | 70.19 (6.05) | 57.81 (11.72) | 88.24 (2.21) | 85.66 (2.82) | 48.14 (6.85) | 79.00 (3.50) |

FAF modality

| | | | | | | | |
|---|---|---|---|---|---|---|---|
| Human level | | | | | | | |
| Fellow | 79.41 (4.83) | 71.43 (4.21) | 78.12 (6.25) | 85.29 (4.41) | — | 67.92 (6.98) | 85.00 (5.00) |
| Attending Other | 68.32 (5.86) | 73.05 (6.69) | 68.75 (10.94) | 86.76 (7.72) | — | 54.84 (9.00) | 81.00 (4.25) |
| Attending Retina | 81.81 (3.43) | 91.25 (12.91) | 70.31 (5.47) | 96.32 (5.88) | — | 75.17 (4.51) | 90.00 (1.75) |
| Overall | 79.41 (12.63) | 74.07 (14.78) | 75.00 (12.50) | 88.24 (8.82) | — | 67.92 (18.85) | 85.00 (8.00) |
| Model level | | | | | | | |
| Standard (non-M3) | 78.51 (8.51) | 92.67 (7.28) | 65.62 (10.16) | 97.79 (2.57) | 94.18 (2.82) | 71.07 (11.98) | 88.50 (5.00) |
| M3 | 85.25 (5.24) | 91.26 (4.52) | 81.25 (2.34) | 96.32 (1.47) | 95.56 (2.24) | 78.79 (7.59) | 91.00 (3.25) |

### Example 16: Attention Maps

For the RPD feature, attention maps were generated to investigate the image locations that contributed most to decision-making by the deep learning models. This was done by back-projecting the last convolutional layer of the neural network. The keras-vis package was used to generate the attention maps.

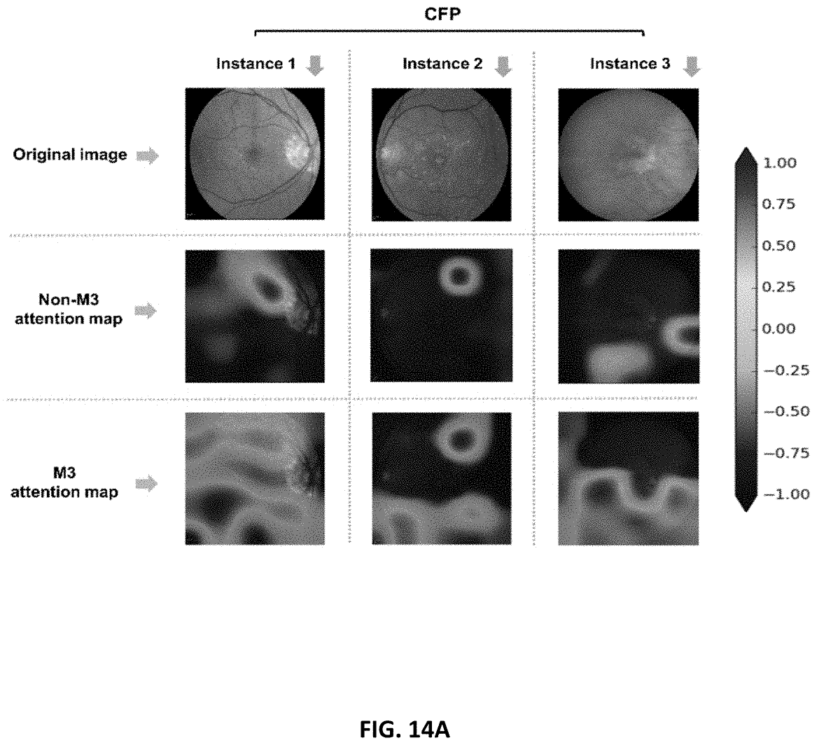

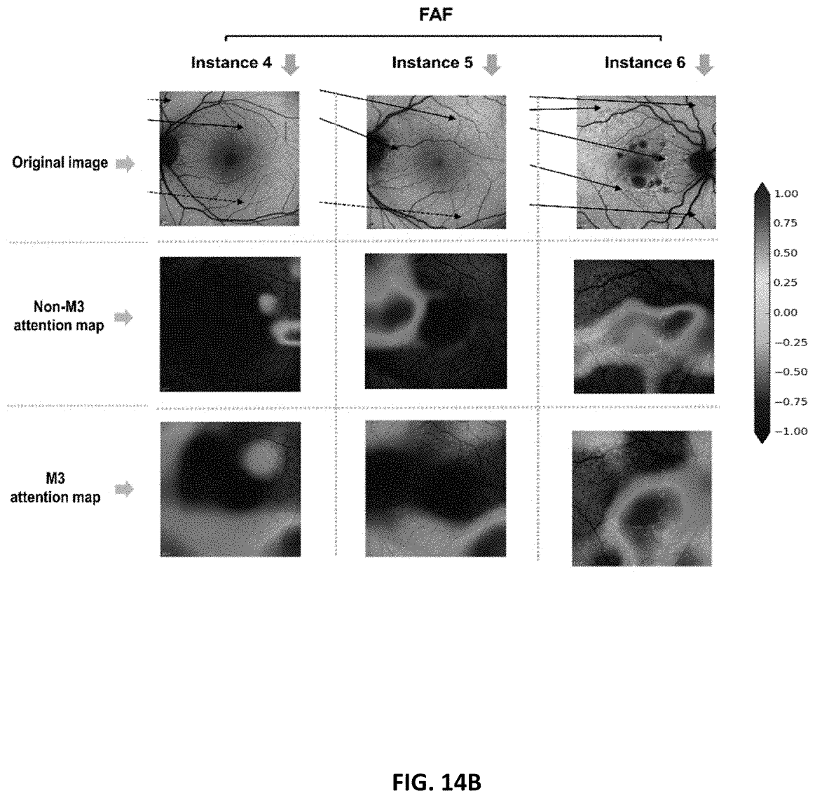

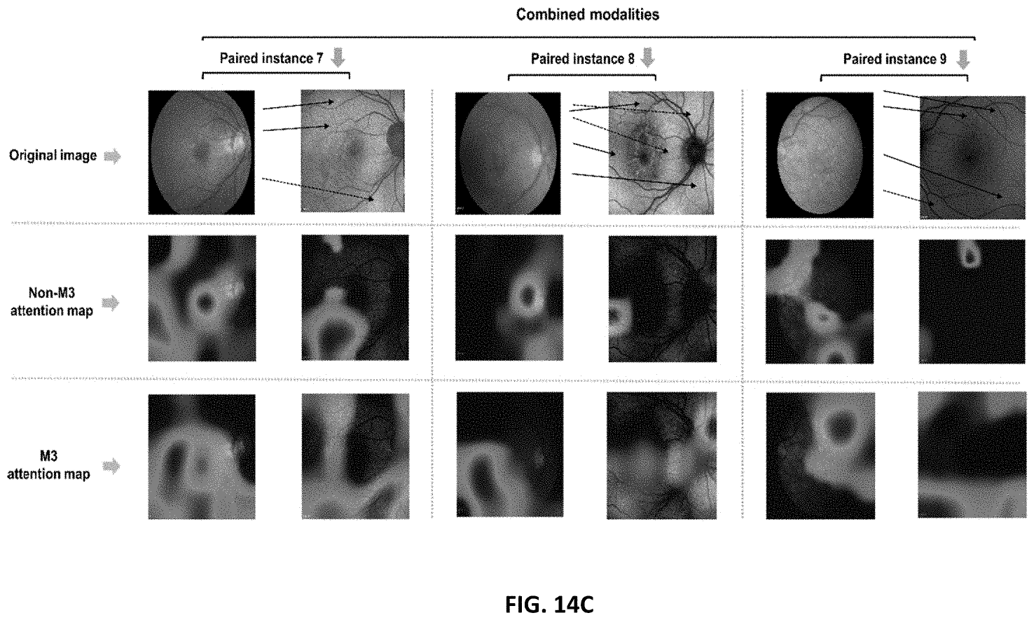

Attention maps were generated and superimposed on the fundus images. For each image, these demonstrate quantitatively the relative contributions made by each pixel to the detection decision. FIGS. 14A-14C shows representative examples where the non-M3 model missed RPD presence but the M3 model correctly detected it. In general, for all three image scenarios, the non-M3 models had only one or very few focal areas of high signal; often, these did not correspond with retinal areas where RPD are typically located. By contrast, the M3 models tended to demonstrate more widespread areas of high signal that corresponded well with retinal areas where RPD are located (e.g. peripheral macula).

### Example 17: External Validation of Deep Learning Models Using a Secondary Dataset not Involved in Model Training

A secondary and separate dataset was used to perform external validation of the trained deep learning models in the detection of RPD. The secondary dataset was the dataset of images, labels, and accompanying clinical information from a previously published analysis of RPD in the Rotterdam Study. In this prior study, eyes with and without RPD were selected from the Rotterdam Study, a prospective cohort study investigating risk factors for chronic diseases in the elderly. The study adhered to the tenets in the Declaration of Helsinki and institutional review board approval was obtained.

The dataset comprised 278 eyes of 230 patients aged 65 years and older, selected from the last examination round of the Rotterdam Study and for whom three image modalities were available (CFP, FAF, and NIR). The positive cases comprised all those eyes in which RPD were detected from CFP (n=72 eyes); RPD presence was confirmed on both FAF and NIR images. The negative cases comprised eyes with soft drusen and no RPD (n=108) and eyes with neither soft drusen nor RPD (i.e. no AMD; n=98); RPD absence was required on all three image modalities (i.e. CFP, FAF, and NIR). The ground truth labels for RPD presence/absence came from human expert graders locally in the Rotterdam Study. RPD were defined as indistinct, yellowish interlacing networks with a width of 125 to 250 μm on CFP; groups of hypoautofluorescent lesions in regular patterns on FAF, and groups of hyporeflectant lesions against a mildly hyperreflectant background in regular patterns on NIR images.

Each deep learning model was evaluated against the gold standard grades on the full set of images (n=278). As for the primary dataset, the performance of the deep learning models was evaluated separately for the three imaging scenarios. The same performance metrics were used as above.

The results are shown in Table 11. The F1-scores of the three M3 models were 78.74 (CFP-alone), 65.63 (FAF-alone), and 79.69 (paired CFP-FAF). The equivalent AUROC values were 96.51, 90.83, and 95.03, respectively. Hence, the performance of the CFP M3 model demonstrated very robust external validation, with performance on the external dataset that was actually substantially higher than on the primary dataset. The F1-score of the FAF M3 model was inferior on the external dataset, and AUROC was modestly inferior. The F1-score of the CFP-FAF M3 model on the external dataset was very similar to that for the primary dataset, and the AUROC was actually superior on the external dataset.

TABLE 11

| | | | Sensitivity | | | | |
|---|---|---|---|---|---|---|---|
| | F1-score | Precision | (Recall) | Specificity | AUROC | Kappa | Accuracy |
| CFP | 78.74 | 94.34 | 67.57 | 98.53 | 96.51 | 72.67 | 90.29 |
| FAF | 65.63 | 77.78 | 56.76 | 94.12 | 90.83 | 55.69 | 84.17 |
| CFP & FAF | 79.69 | 94.44 | 68.92 | 98.53 | 95.03 | 73.80 | 90.65 |

### Example 18: Automated Detection of Geographic Atrophy and Pigmentary Abnormalities by Multi-Modal, Multi-Task, Multi-Attention (M3) Deep Learning Models

M3 deep learning models were trained to detect two other important features of AMD, geographic atrophy and pigmentary abnormalities. For the detection of geographic atrophy, in all three image scenarios, the median F1-scores of the M3 models were numerically higher than those of the non-M3 models. The differences were statistically significant for the CFP-only and FAF-only scenarios (p<0.001 and p<0.01, respectively). The superiority of the M3 models was particularly evident for the clinically important CFP-only scenario. In the CFP-only scenario, the median F1-score was 83.99 (IQR 1.80) for the M3 model and 80.20 (IQR 1.48) for the non-M3 model. The model with the highest F1-score was the M3 model in the CFP-FAF scenario, at 85.45 (IQR 1.24).

For the detection of pigmentary abnormalities, again, in all three image scenarios, the median F1-scores of the M3 models were numerically higher than those of the non-M3 models. The differences were statistically significant for the FAF-only and CFP-FAF scenarios (p<0.05 and p<0.0001, respectively). The model with the highest F1-score was the M3 model in the CFP-FAF scenario, at 88.79 (IQR 0.50).

Having described several embodiments, it will be recognized by those skilled in the art that various modifications, alternative constructions, and equivalents may be used without departing from the spirit of the disclosure. Additionally, a number of well-known processes and elements have not been described in order to avoid unnecessarily obscuring the present disclosure. Accordingly, the above description should not be taken as limiting the scope of the disclosure.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A method of predicting risk of late age-related macular degeneration (AMD), the method comprising:
receiving one or more color fundus photograph (CFP) images from both eyes of a patient;
classifying each CFP image, wherein classifying each CFP image comprises:
extracting one or more deep features in each CFP image; or
grading drusen and pigmentary abnormalities; and
predicting the risk of late AMD by estimating a time to late AMD using a Cox proportional hazard model using the one or more deep features or the graded drusen and pigmentary abnormalities.

2. The method of claim 1, wherein predicting the risk of late AMD comprises predicting the risk of geographic atrophy (GA).

3. The method of claim 1, wherein predicting the risk of late AMD comprises predicting the risk of neovascular AMD (NV).

4. The method of claim 1, wherein the one or more deep features comprise macular drusen and pigmentary abnormalities.

5. The method of claim 1, further comprising normalizing the one or more deep features as a standard score.

6. The method of claim 1, further comprising receiving demographic and/or genotype information of the patient selected from patient age, smoking status, and/or AMD genotype.

7. The method of claim 6, wherein the AMD genotype is selected from CFH rs1061170, ARMS2 rs10490924, and the AMD GRS.

8. The method of claim 1, wherein each CFP image is classified automatically.

9. The method of claim 1, wherein the risk of late AMD is predicted automatically.

10. The method of claim 1 further comprising detecting the presence of reticular pseudodrusen (RPD) in one or more of the CFP images.

11. A method of predicting risk of late AMD, the method comprising,
receiving one or more images from both eyes of a patient;
classifying each image, wherein classifying each image comprises:
detecting the presence of RPD in each image; and
predicting the risk of late AMD by estimating a time to late AMD using the presence of RPD in each image.

12. The method of claim 11, wherein the one or more images is selected from color fundus photograph (CFP) images, fundus autofluorescence (FAF) images, and/or near-infrared images.

13. The method of claim 11, wherein the presence of RPD is detected automatically.

14. The method of claim 11, wherein the one or more images are CFP images and wherein classifying each image further comprises:
extracting one or more deep features in each CFP image; or
grading the drusen and pigmentary abnormalities.

15. The method of claim 14, wherein predicting the risk of late AMD comprises estimating a time to late AMD using a Cox proportional hazard model using the presence of RPD, the one or more deep features, and/or the graded drusen and pigmentary abnormalities.

16. A device comprising at least one non-transitory computer readable medium storing instructions which when executed by at least one processor, cause the at least one processor to:
receive one or more color fundus photograph (CFP) images from both eyes of a patient;
classify each CFP image, wherein classifying each CFP image comprises:
extract one or more deep features for macular drusen and pigmentary abnormalities in each CFP image;
grade the drusen and pigmentary abnormalities; and/or
detect the presence of RPD in each CFP image; and
predict the risk of late AMD by estimating a time to late AMD using a Cox proportional hazard model using the presence of RPD, the one or more deep features, and/or the graded drusen and pigmentary abnormalities.

17. The device of claim 16, wherein predicting the risk of late AMD comprises predicting the risk of GA.

18. The device of claim 16, wherein predicting the risk of late AMD comprises predicting the risk of NV.

19. The device of claim 16, wherein the one or more deep features comprise macular drusen and/or pigmentary abnormalities.

20. The device of claim 16, further comprising instructions, which when executed by the at least one processor, cause the at least one processor to normalize the one or more deep features as a standard score.

21. The device of claim 16, further comprising receiving demographic and/or genotype information of the patient selected from patient age, smoking status, and/or AMD genotype.

22. The device of claim 16, wherein each CFP image is classified automatically and the risk of late AMD is predicted automatically.
